SEQUENCE LISTING (40 sequences)

SEQ ID NO: 1 - protein, 374 aa, unidentified
SGVFQLQLQEFINERGVLASGRPCEPGCRTFFRVCLKHFQAVVSPGPCTFGTVSTPVLGTNSFAVRDDSSGGGRNPLQLPFNFTWPGTFSLIIEAWHAPGDDLRPEALPPDALISKIAIQGSLAVGQNWLLDEQTSTLTRLRYSYRVICSDNYYGDNCSRLCKKRNDHFGHYVCQPDGNLSCLPGWTGEYCQQPICLSGCHEQNGYCSKPAECLCRPGWQGRLCNECIPHNGCRHGTCSTPWQCTCDEGWGGLFCDQDLNYCTHHSPCKNGATCSNSGQRSYTCTCRPGYTGVDCELELSECDSNPCRNGGSCKDQEDGYHCLCPPGYYGLHCEHSTLSCADSPCFNGGSCRERNQGANYACECPPNFTGSNCE

SEQ ID NO: 2 - protein, 374 aa, unidentified
SSVFQLQLQEFINERGVLASGRPCEPGCRTFFRVCLKHFQAVVSPGPCTFGTVSTPVLGTNSFAVRDDSSGGGRNPLQLPLNFTWPGTFSLIIEAWHAPGDDLRPEALPPDALISKFAIQGSLAVGQNWLLDEQTSTLTRLRYSYRVICSDNYYGDNCSRLCKKRNDYFGHYVCQPDGNPSCLPGWTGEYCQQPICLSGCHEQNGYCSKPAECLCRPGWQGRLCNECIPHNGCRHGTCSTPWQCTCDEGWGGLFCDQDLNYCTHHSPCKNGATCSNSGQRSYTCTCRPGYTGVDCELELSECDSNPCRNGGSCKDQEDGYHCLCPPGYYGLHCEHSTLSCADSPCFNGGSCRERNQGANYACECPPNFTGSNCE

SEQ ID NO: 3 - protein, 374 aa, unidentified
SSVFQLQLQEFINERGVLASGRPCEPGCRTFFRVCLKHFQAVVSPGPCTFGTVSTPVLGTNSFAVRDDSSGGGRNPLQLPLNFTWPGTFSLIIEAWHAPGDDLRPEALPPDALISKFAIQGSLAVGQNWLLDEQTSTLTRLRYSYRVICSDNYYGDNCSRLCKKRNDYFGHYVCQPDGNPSCLPGWTGEYCQQPICLSGCHEQNGYCSKPAECLCRPGWQGRLCNECIPYPGCRHGTCSTPWQCLCDEGWGGLYCDQDLNYCTHHSPCKNGATCHNSGPRSYTCTCRPGYTGVDCELELSECDSNPCRNGGSCKDQEDGYHCLCPPGYYGLHCEHSTLSCADSPCFNGGSCRERNQGANYACECPPNFTGSNCE

SEQ ID NO: 4 - protein, 374 aa, unidentified
SSVFQLQLQEFINERGVLASGRPCEPGCRTFFRVCLKHFQAVVSPGPCTFGTVSTPVLGTNSFAVRDDSSGGGRNPLQLPLNFTWPGTFSLIIEAWHAPGDDLRPEALPPDALISKFAIQGSLAVGQNWLLDEQTSTLTRLRYSYRVICSDNYYGDNCSRLCKKRNDYFGHYVCQPDGNPSCLPGWTGEYCQQPICLSGCHEQNGYCSKPAECLCRPGWQGRLCNECIPHPGCRHGTCSTPWQCLCDEGWGGLYCDQDLNYCTHHSPCKNGATCRNSGPRSYTCTCRPGYTGVDCELELSECDSNPCRNGGSCKDQEDGYHCLCPPGYYGLHCEHSTLSCADSPCFNGGSCRERNQGANYACECPPNFTGSNCE

SEQ ID NO: 5 - protein, 373 aa, unidentified
SGVFQLQLQEFINERGVLASGRPCEPGCRTFFRVCLKHFQAVVSPGPCTFGTVSTPVLGTNSAVRDDSSGGGRNPLQLPFNFTWPGTFSLIIEAWHAPGDDLRPEALPPDALISKIAIQGSLAVGQNWLLDEQTSTLTRLRYSYRVICSDNYYGDNCSRLCKKRNDHFGHYVCQPDGNLSCLPGWTGEYCQQPICLSGCHEQNGYCSKPAECLCRPGWQGRLCNECIPHPGCRHGTCSTPWQCLCDEGWGGLYCDQDLNYCTHHSPCKNGATCRNSGPRSYTCTCRPGYTGVDCELELSECDSNPCRNGGSCKDQEDGYHCLCPPGYYGLHCEHSTLSCADSPCFNGGSCRERNQGANYACECPPNFTGSNCE

SEQ ID NO: 6 - protein, 374 aa, unidentified
SGVFQLQLQEFINERGVLASGRPCEPGCRTFFRVCLKHFQAVVSPGPCTFGTVSTPVLGTNSFAVRDDSSGGGRNPLQLPFNFTWPGTFSLIIEAWHAPGDDLRPEALPPDALISKIAIQGSLAVGQNWLLDEQTSTLTRLRYSYRVICSDNYYGDNCSRLCKKRNDHFGHYVCQPDGNLSCLPGWTGEYCQQPICLSGCHEQNGYCSKPAECLCRPGWQGRLCNECIPYPGCRHGTCSTPWQCLCDEGWGGLYCDQDLNYCTHHSPCKNGATCHNSGPRSYTCTCRPGYTGVDCELELSECDSNPCRNGGSCKDQEDGYHCLCPPGYYGLHCEHSTLSCADSPCFNGGSCRERNQGANYACECPPNFTGSNCE

SEQ ID NO: 7 - protein, 374 aa, unidentified; identical to SEQ ID NO: 1

SEQ ID NO: 8 - protein, 374 aa, unidentified; identical to SEQ ID NO: 2

SEQ ID NO: 9 - protein, 374 aa, unidentified; identical to SEQ ID NO: 3

SEQ ID NO: 10 - protein, 374 aa, unidentified; identical to SEQ ID NO: 4

SEQ ID NO: 11 - protein, 373 aa, unidentified; identical to SEQ ID NO: 5

SEQ ID NO: 12 - protein, 374 aa, unidentified; identical to SEQ ID NO: 6

SEQ ID NO: 13 - protein, 9 aa, unidentified
YPYDVPDYA

SEQ ID NO: 14 - protein, 10 aa, unidentified
EQKLISEEDL

SEQ ID NO: 15 - DNA, 18 bp, synthetic construct
  source 1..18: mol_type = other DNA
aaatgataaccatctcgc

SEQ ID NO: 16 - DNA, 22 bp, synthetic construct
  source 1..22: mol_type = other DNA
gggatttgctcgcatatagttg

SEQ ID NO: 17 - DNA, 21 bp, synthetic construct
  source 1..21: mol_type = other DNA
atgagtatgcctgccgtgtga

SEQ ID NO: 18 - DNA, 20 bp, synthetic construct
  source 1..20: mol_type = other DNA
ggcatcttcaaacctccatg

SEQ ID NO: 19 - DNA, 20 bp, synthetic construct
  source 1..20: mol_type = other DNA
gaccagagcatccaaaagag

SEQ ID NO: 20 - DNA, 25 bp, synthetic construct
  source 1..25: mol_type = other DNA
ggacattcaagtcagttaccgaata

SEQ ID NO: 21 - DNA, 21 bp, synthetic construct
  source 1..21: mol_type = other DNA
ccctgggaaaacactcacaca

SEQ ID NO: 22 - DNA, 22 bp, synthetic construct
  source 1..22: mol_type = other DNA
gcacaactcaatggtactgtcg

SEQ ID NO: 23 - DNA, 19 bp, synthetic construct
  source 1..19: mol_type = other DNA
acggtcatctccaggatgt

SEQ ID NO: 24 - DNA, 20 bp, synthetic construct
  source 1..20: mol_type = other DNA
cgagcgcgtattaacgagag

SEQ ID NO: 25 - DNA, 21 bp, synthetic construct
  source 1..21: mol_type = other DNA
agggctttcttgggtcagagg

SEQ ID NO: 26 - DNA, 21 bp, synthetic construct
  source 1..21: mol_type = other DNA
gctgcgaatcttcagcactca

SEQ ID NO: 27 - DNA, 21 bp, synthetic construct
  source 1..21: mol_type = other DNA
ccaagtgtggctcattaggca

SEQ ID NO: 28 - DNA, 22 bp, synthetic construct
  source 1..22: mol_type = other DNA
ccaatcttcgactggactctgt

SEQ ID NO: 29 - DNA, 20 bp, synthetic construct
  source 1..20: mol_type = other DNA
accagttggctgatgagtcg

SEQ ID NO: 30 - DNA, 23 bp, synthetic construct
  source 1..23: mol_type = other DNA
caaagtcctgataccagcatctt

SEQ ID NO: 31 - DNA, 19 bp, synthetic construct
  source 1..19: mol_type = other DNA
tggacagttacgcgcacat

SEQ ID NO: 32 - DNA, 21 bp, synthetic construct
  source 1..21: mol_type = other DNA
cgagtaggacatgctgtaggt

SEQ ID NO: 33 - DNA, 21 bp, synthetic construct
  source 1..21: mol_type = other DNA
gctgctggtagggatcattca

SEQ ID NO: 34 - DNA, 23 bp, synthetic construct
  source 1..23: mol_type = other DNA
gtttttcggtggacttttgtctc

SEQ ID NO: 35 - DNA, 23 bp, synthetic construct
  source 1..23: mol_type = other DNA
acaattttcctgactccaaggac

SEQ ID NO: 36 - DNA, 21 bp, synthetic construct
  source 1..21: mol_type = other DNA
actgtgacgaagctcagagac

SEQ ID NO: 37 - DNA, 20 bp, synthetic construct
  source 1..20: mol_type = other DNA
gggagacaatcgagctgacg

SEQ ID NO: 38 - DNA, 19 bp, synthetic construct
  source 1..19: mol_type = other DNA
tgcagcggataccgaagga

SEQ ID NO: 39 - DNA, 22 bp, synthetic construct
  source 1..22: mol_type = other DNA
gagctggttcagaaggccaaac

SEQ ID NO: 40 - DNA, 26 bp, synthetic construct
  source 1..26: mol_type = other DNA
ccttgctcagttacagacttcatgca

(19) United States
(12) Patent Application Publication
(10) Pub. No.: US 2026/0055369 A1
(43) Pub. Date: Feb. 26, 2026

(54) METHOD FOR GENERATING CELLS OF THE T CELL LINEAGE WITH ENGINEERING BROADLY REACTIVE HUMAN NOTCH LIGAND

(71) Applicant: H. Lee Moffitt Cancer Center and Research Institute, Inc., Tampa, FL (US)

(72) Inventor: Vincent C. LUCA, Tampa, FL (US)

(21) Appl. No.: 19/103,225

(22) PCT Filed: Aug. 7, 2023
(86) PCT No.: PCT/US2023/071775

§ 371 Date: Feb. 11, 2025

Related U.S. Application Data
(60) Provisional application No. 63/371,176, filed on Aug. 11, 2022.

(51) Int. Cl. C12N 5/0783

(52) U.S. Cl. CPC C12N 5/0636; C12N 2501/42; C12N 2531/00

(57) ABSTRACT

A method of generating cells of the T cell lineage is provided that involves culturing a sample comprising stem cells or progenitor cells with an engineered Notch ligand conjugated to a suspension support and isolating cells of the T cell lineage. In one embodiment, the cells of the T-cell lineage are progenitor T cells or mature T cells. Compositions, kits and uses thereof are also provided.

### CROSS-REFERENCE TO RELATED APPLICATIONS

This application claims benefit of U.S. Provisional Application No. 63/371,176, filed Aug. 11, 2022, which is hereby incorporated herein by reference in its entirety.

### SEQUENCE LISTING

This application contains a sequence listing filed in ST.26 format entitled “320803_2920_Sequence_Listing” created on Aug. 7, 2023, having 40,235 bytes. The content of the sequence listing is incorporated herein in its entirety.

### BACKGROUND

T cells are critical mediators of adaptive immunity and can be harnessed as therapeutic agents against pathogens and in cancer immunotherapy. Hematopoietic stem cell transplantation (HSCT) offers an effective treatment for a broad spectrum of malignant and non-malignant disorders, but the preconditioning regimens required before treatment results in an extended delay in T cell recovery (Krenger et al., 2011). In contrast to most other hematopoietic lineages, which develop in the bone marrow (BM), T cell development requires the migration of BM-derived progenitors to the thymus, wherein the incoming lymphocyte progenitors receive critical signals to induce their differentiation into T-lineage cells (Shah and Zuniga-Pflucker, 2014).

In the context of HSCT, thymus dysfunction or atrophy, as a result of conditioning and aging, respectively, combined with a limited capacity of transplanted HSCs to give rise to lymphocytes restrain the extent of T cell development in the thymus (Porter and June 2005). This leads to inadequate immune surveillance, predisposing patients to infections and/or relapse of cancer and remains a serious clinical challenge.

The adoptive transfer of progenitor T (proT) cells has emerged as a promising strategy for enhancing T cell reconstitution, as human or mouse proT cells have been shown to engraft the thymus of immunodeficient mice despite their xenogeneic or allogeneic origin (Awong et al., 2009; Awong et al., 2013; Zakrzewski et al., 2006; Zakrzewski et al., 2008). ProT cells are developmentally immature and undergo positive and negative selection in the host thymus. Thus, they become restricted to the recipient's major histocompatibility complex (MHC) yielding host tolerant T cells that can bypass the clinical challenges associated with graft-versus-host disease (GVHD). Importantly, engraftment with proT cells restores the thymic architecture and improves subsequent thymic seeding by HSC-derived progenitors. In addition to its intrinsic regenerative medicine properties, proT cells can also be engineered with T cell receptors (TCRs) and chimeric antigen receptors (CARs) to confer specificity to tumor-associated antigens (TAA) to treat cancer and also with synthetic gene circuits to sculpt custom response programs.

An unmet challenge in the field is the development of a clinically relevant system that could be easily scaled up to generate large quantities of proT cells from different sources of human hematopoietic stem/progenitor cells (HSPC). Previous methods have relied on mouse-derived OP9 cells expressing the Notch ligands Delta-like-1 (DL1) or Delta-like-4 (DL4), however, this approach presents several challenges for clinical translation (Awong et al., 2009; Awong et al., 2013). Most strategies for a stromal cell-free approach have relied on a two-dimensional (2D) tissue culture platform, whereby Notch ligands, DL1 or DL4, are immobilized onto tissue-culture plates (Gehre et al., 2015; Reimann et al., 2012; Simons et al., 2017). Additional adhesion receptor ligands, like vascular cell adhesion molecule-1 (VCAM-1), have also been included in this format (Shukla et al., 2017). Human proT cells produced using these strategies have been shown to successfully reconstitute the thymus of immunodeficient mice. While the progress is encouraging, the utility of these approaches to generate proT cells for therapy is limited due to the need for scale-up processing for clinical manufacture and not an effective method for routine generation of large-scale cell numbers to be clinically applicable. Ideally, a truly scalable platform would allow for proT cells to be grown in closed automated bioreactor systems (Lipsitz et al., 2016).

### SUMMARY

Disclosed herein is a cell-free, bead-based system for generating cells of the T cell lineage from mouse or human hematopoietic stem/progenitor cells (HSPCs) and induced pluripotent stem cells (iPSCs). Non-plate-bound suspensions of Notch ligands (for example DL4-μbeads) allow for the effective generation of T-lineage cells including progenitor T cells and mature T cells.

Accordingly, the disclosure provides a method of generating a cell of the T cell lineage comprising (a) culturing a sample comprising stem cells or progenitor cells with an engineered Notch ligand disclosed herein conjugated to a suspension support and (b) isolating cells of the T cell lineage.

In some embodiments, the engineered Notch ligand is an engineered DLL4 protein comprising a conservative amino acid substitution at a residue corresponding to residues 28, 107, 143, 194, and 206 as set forth in SEQ ID NO: 1 or SEQ ID NO: 7 and further comprising at least one conservative amino acid substitution at residues 256, 257, 271, 280, 301, and 305 as set forth in SEQ ID NO: 1 or SEQ ID NO: 7.

In some embodiments, the suspension support is a particle. In some embodiments, the suspension support is a microbead.

In some embodiments, the stem cells or progenitor cells with the engineered Notch ligands are cultured in suspension. In some embodiments, the stem cells are selected from hematopoietic stem/progenitor cells (HSPCs), embryonic stem cells or induced pluripotent stem cells (iPSCs). In some embodiments, the stem cells are human stem cells, optionally CD34+ or CD34+ CD38−/lo HSPCs. In some embodiments, the stem cells are CD34+ hematopoietic precursor cells, optionally CD34+ hematopoietic precursor cells that have been differentiated from iPSCs.

In some embodiments, the cells of the T cell lineage are progenitor T (proT) cells. In some embodiments, the stem cells or progenitor cells are human cells and the proT cells have the phenotype CD34+ CD7+ or CD7+ CD5+ CD1a−. In some embodiments, the stem cells or progenitor cells are mouse cells, optionally lineage—CD117+ Sca-1+ mouse cells, and the proT cells have the phenotype CD25+ or CD25+ CD90+. In some embodiments, the cells of the T-cell lineage are CD4+ CD8+ double positive cells, CD4+ CD8+ CD3+ double positive cells, CD8+ CD3+ single positive cells or CD4+ CD3+ single positive cells.

In some embodiments, the stem cells or progenitor cells are cultured in stromal cell-free media. In some embodiments, the stem cells or progenitor cells are cultured with at least one T cell co-stimulatory molecule attached to a suspension support, optionally wherein the at least one T cell co-stimulatory molecule is VCAM1.

The disclosure also provides a cell of the T cell lineage, wherein the cell is generated by a method comprising: (a) culturing a sample comprising stem cells or progenitor cells with an engineered Notch ligand disclosed herein conjugated to a suspension support and (b) isolating cells of the T cell lineage.

In one embodiment, the cell is a progenitor T cell, CD4+ CD8+ double positive cell, CD4+ CD8+ CD3+ double positive cell or CD8+ CD3+ single positive cell, CD4+ CD3+ single positive cell.

Also disclosed herein is a suspension of engineered Notch ligand disclosed herein comprising (a) a Notch ligand and (b) a suspension support, optionally a microbead, wherein the engineered Notch ligand is conjugated to the suspension support.

In some embodiments, the suspension support is a microbead. In some embodiments, the microbead has a diameter of 1 to 10 μm, 1 to 20 μm, 1 to 30 μm, 5 to 10 μm, 5 to 20 μm, 5 to 100 μm, or 20 to 30 μm, In some embodiments, the C-terminus region of the engineered Notch ligand is conjugated to the microbead.

Also disclosed herein is a use of the suspension Notch ligand for generating cells of the T cell lineage.

Also disclosed herein is a method of treating a subject having a condition requiring an increase in the number of T cells comprising:
(i) generating cells of the T cell lineage comprising (a) culturing a sample comprising stem cells or progenitor cells with an engineered Notch ligand disclosed herein conjugated to a suspension support and (b) isolating cells of the T cell lineage, and
(ii) administering an effective amount of the cells of the T cell lineage to the subject.

In some embodiments, the cells of the T cell lineage are progenitor T cells. In some embodiments, the cells of the T cell lineage are mature T cells. In some embodiments, the cells of the T cell lineage are CD4+CD8+ double positive cells, CD4+CD8+CD3+ double positive cells, CD8+CD3+ single positive cells or CD4+CD3+ single positive cells.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1. Yeast display selection strategy for affinity-maturation of human DLL4. a, Cartoon schematic of the design strategy for the DLL4 Site 3 mutant library. Red spheres depict mutated interface residues. b, Table depicting DLL4 interface residue positions and the mutations allowed at each position and schematic of DLL4 yeast display construct. Yellow stars indicate mutations. c, Flow cytometry histogram plots of yeast stained with fluorescently-labeled Notch3 protein following each round of selection (left), and a table indicating the frequency of mutations and consensus DLL4 mutant sequence (right). Histograms are representative of three biological replicates. d, Structural models highlighting the mutated residues in DLL4.v2. WT residues in DLL4 and mutated residues in DLL4.v2 are colored orange and shown in surface representation. DLL4 models were based on DLL4:Notch1 (PDB ID: 4XL1, for C2-DSL-EGF1 domains) and JAG1:Notch1 (PDB ID: 5UK5, for EGF1-EGF3) structures.

FIG. 2. Biophysical characterization of the Delta^{MAX} variant. a, Schematic depicting the generation of Delta^{MAX} through engraftment of DLL4.v1 and DLL4.v2 mutations. Red spheres indicate affinity-enhancing mutations. b, SPR binding isotherms measuring DLL4 variant interactions with the ligand binding domains of Notch1-4. The table shows the K_{D} determined for each interaction and the fold-enhancement of Delta^{MAX} affinity relative to WT DLL4. c, Representative SPR sensograms depicting the binding of 200 nM concentrations of WT DLL4, DLL4.v1, DLL4.v2, and Delta^{MAX} to Notch1 EGF6-13. d, DSF was used to determine the Tₘ of WT DLL4 and Delta^{MAX}. Data represent mean values±s.d. of three replicates.

FIG. 3. Delta^{MAX} is more potent Notch agonist than WT DLL4. a, Dose-titration assay comparing the Notch1 reporter activity stimulated by WT DLL4 and Delta^{MAX} proteins that were non-specifically adsorbed to tissue culture plates. b, Dose-titration assay comparing the Notch1 reporter activity stimulated by WT DLL4 and Delta^{MAX} proteins. DLL4 ligands were C-termini biotinylated and coupled to streptavidin-coated plates. c, Time-course experiment comparing Notch1 reporter activity stimulated by WT DLL4 or Delta^{MAX} immobilized at 50 nM. d, A luciferase reporter assay was used to measure Notch1, Notch2, or Notch3 activation by WT DLL4 and Delta^{MAX}. Protein concentrations were a 10-fold serial dilution from 50 nM to 0.05 nM. e, Detection of endogenous cleaved Notch intracellular domain (N1ICD) in U2OS and MCF-7 cell lines by Western blot. Cells were Notch activated with 50 nM of WT DLL4 and Delta^{MAX} immobilized in streptavidin plates. f, Co-culture assay comparing Notch1 activation by Delta^{MAX} and WT DLL4. Notch1 reporter cells and ligand-expressing cells were cultured at a 1:1 ratio. g, Notch1 luciferase reporter cells were stimulated with Delta^{MAX} protein in different formats. Yeast expressing Delta^{MAX} were co-cultured in 10:1 ratio with reporter cells; HEK293T cells stably expressing Delta^{MAX} were co-cultured in 1:1 ratio; Delta^{MAX}-coated streptavidin beads were mixed in 20:1 ratio; 0.1 ng/mm² Delta^{MAX} was non-specifically adsorbed to surfaces prior to addition of reporter cells; 10 nM C-terminally biotinylated Delta^{MAX} was immobilized on streptavidin-coated plates. Notch activation was normalized to the corresponding controls. Delta^{MAX} statistics are referred to WT DLL4. *P<0.05, **P<0.01, ***P<0.001, ****P<0.0001 (Two-way ANOVA). Data represent mean values±s.d. of three biological replicates. Western blot images are representative of three replicates.

FIG. 4. Delta^{MAX} is a pan-Notch inhibitor. a, Dose-titration assay measuring the inhibition of Notch1 reporter activity by soluble DLL4 variants. Notch1 signaling was activated by culturing cells on streptavidin plates coated with 50 nM WT DLL4. DLL4.v1, DLL4.v2 statistics are referred to WT DLL4. b, Dose-titration assay comparing the inhibition potency of DAPT, BB-94, and soluble Delta^{MAX}. Notch1 signaling was activated by culturing cells on streptavidin plates coated with 50 nM WT DLL4. DAPT statistics are referred to BB-94. c, Soluble WT DLL4 and Delta^{MAX} at 3 μM were tested for their ability to inhibit Notch1 activation by 293T cells overexpressing DLL4, DLL1, JAG1, or JAG2. d, Dose-titration assay comparing the ability of WT DLL4 and Delta^{MAX} to inhibit activation of Notch1-3 by WT DLL4. 10-fold serial dilutions starting at 3,000 nM of soluble DLL4 or Delta^{MAX} were added to Notch1, Notch2, and Notch3 luciferase reporter cells. Notch signaling was activated by culturing cells on streptavidin plates coated with 50 nM WT DLL4. All Delta^{MAX} statistics are referred to WT DLL4. *P<0.05, **P<0.01, ***P<0.001, ****P<0.0001 (Two-way ANOVA). Data represent mean values±s.d. of three biological replicates.

FIG. 5. Delta^{MAX} stimulation increases proliferation and expression of activation markers in human CD8⁺ T cells. a, Human PBMCs CD8⁺ T cells were co-cultured with K32 artificial antigen-presenting cells (aAPCs) engineered to express WT DLL4 or Delta^{MAX} for simultaneous Notch and TCR stimulation. b, aAPCs were assayed for expression of WT DLL4 or Delta^{MAX} using flow cytometry. K32 cells were loaded with OKT3 antibody using a gradient of concentrations starting at 0.0125 μg/ml, and then co-cultured with CD8⁺ T cells in ratio 10:1. After 96 h, CD8⁺ T cells were analyzed for proliferation (c) and IFNγ secretion (d). e, T cells were stimulated for 24, 48, and 96 h to quantify mRNA levels of intracellular activation markers by real-time PCR, including IFNγ, Granzyme B and Hes-4. f, K32 cells expressing WT DLL4 or Delta^{MAX} were co-culture with human CD8⁺ T cells (ratio 10:1) for detection of Notch activation by Western blot. Co-cultured CD8⁺ T cells were positively sorted by MACS after cultivation with K32 cells, and nuclear extracts were harvested after 96 hours. Cleavage of Notch1 and Notch2 were examined by Western blot with specific antibodies. β-actin was used for loading control. Western blot images are representative results of three biological repeats. NA: non-activated T cells. Statistics for Delta^{MAX} are referred to WT DLL4. *P<0.05, **P<0.01, ***P<0.001, ****P<0.0001 (Two-way ANOVA). T cell proliferation and IFNγ secretion data were analyzed by unpaired t tests. Data represent mean values±s.e.m. of three biological replicates.

FIG. 6. Soluble Delta^{MAX} promotes neuronal differentiation. a, Human Pluripotent Stem Cells (hPSC) are differentiated into neuronal progenitors, which maintain its stem cell status through Notch activation. When Notch is inhibited in neuronal progenitors, cells are differentiated into cerebral cortex neurons. A set of multiple differentiation markers (b, c, d, e, f, and g) were measured by qPCR after treatment with soluble WT DLL4, Delta^{MAX}, DAPT (differentiation control), or absence of inhibitor. DLL4 was analyzed at 10, 100, and 1,000 nM, while DAPT was at 10,000 nM. Statistics for Delta^{MAX} are referred to WT DLL4, and DAPT to Delta^{MAX}. *P<0.05, **P<0.01, ***P<0.001, ****P<0.0001 (Two-way ANOVA). Data represent mean values±s.e.m. of three biological replicates.

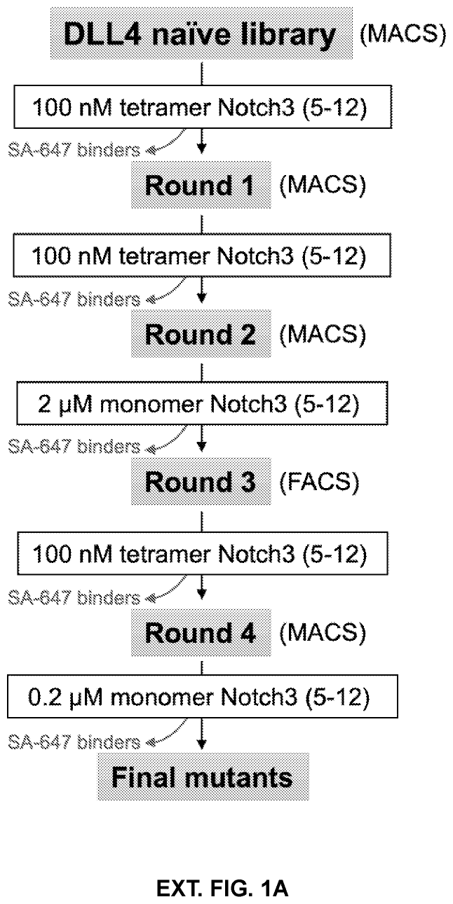

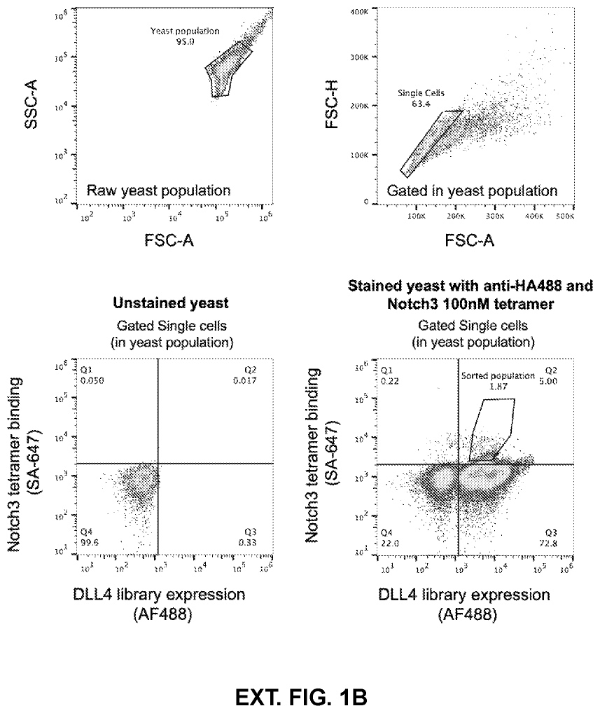

Extended Data FIG. 1. Selection strategy for isolation of affinity-enhancing mutations in DLL4. a, Flow chart depicting the selection strategy used to isolate high-affinity DLL4 variants. Red arrows and text indicate negative selections. b, Gating strategy for sorting high-affinity binders to Notch3.

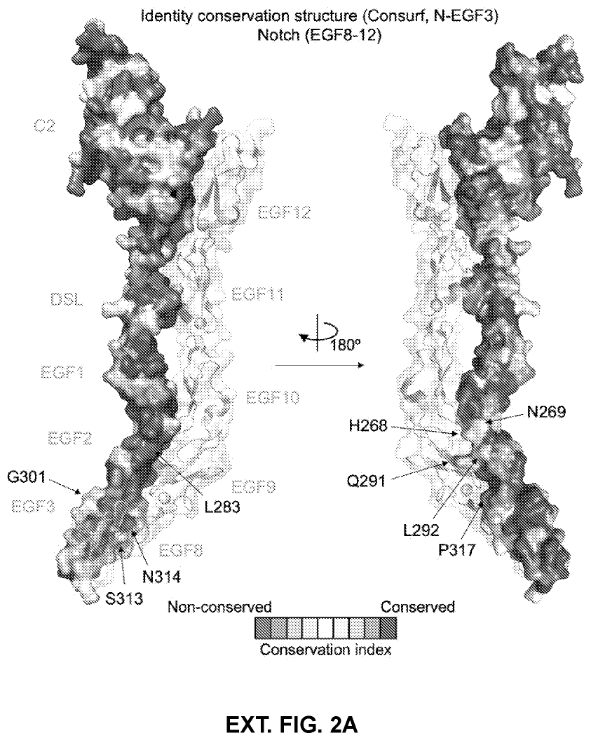

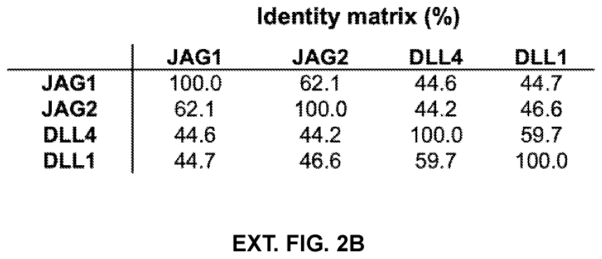

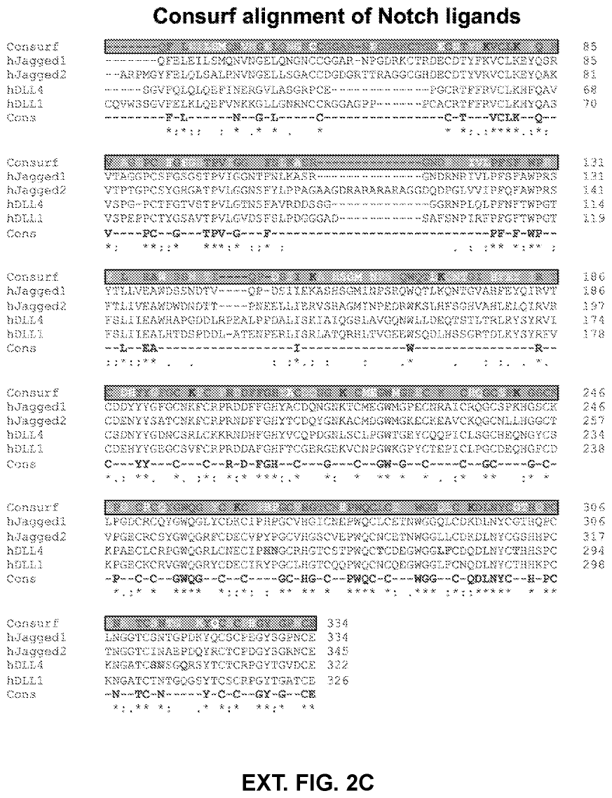

Extended Data FIG. 2. Conservation analysis of Site 3 library positions in activating Notch ligands. a, A structural model depicting amino acid conservation in DLL1, DLL4, JAG1, and JAG2 residues was generated in Consurf using rat JAG1 as a template (PDB ID: 5UK5). b, Identity matrix indicating the sequence identity between human Notch ligands. c, Sequence alignment depicting conservation at each residue position. Residues mutated in the DLL4 mutant library are highlighted in red.

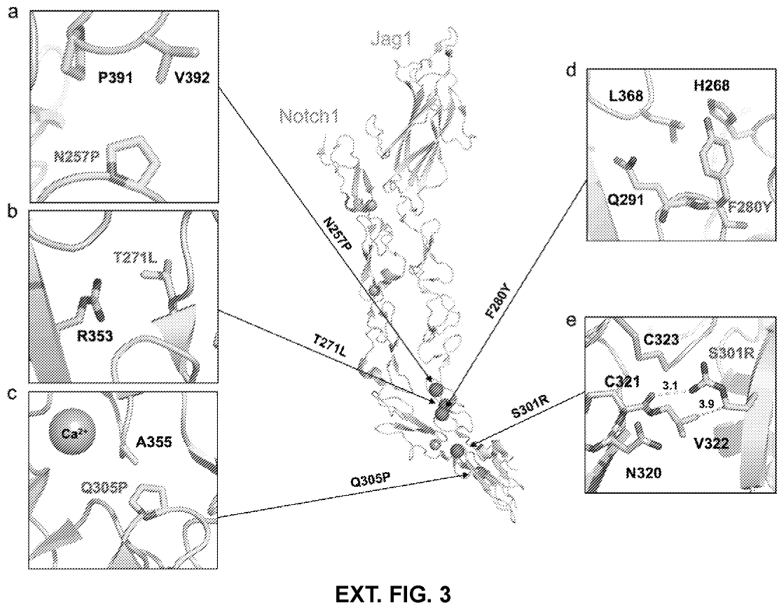

Extended Data FIG. 3. Structural analysis of Delta^{MAX} mutations. Cartoon representation showing the structural context of DLL4. v2 mutant in a model of the rat JAG1-Notch1 complex (PDB ID: 5UK5). Panels a, b, c, d, and e are zoom panels depicting the residues that surround the mutated position. Numbers and dashes in e are inter-atomic distances atoms measured in angstroms.

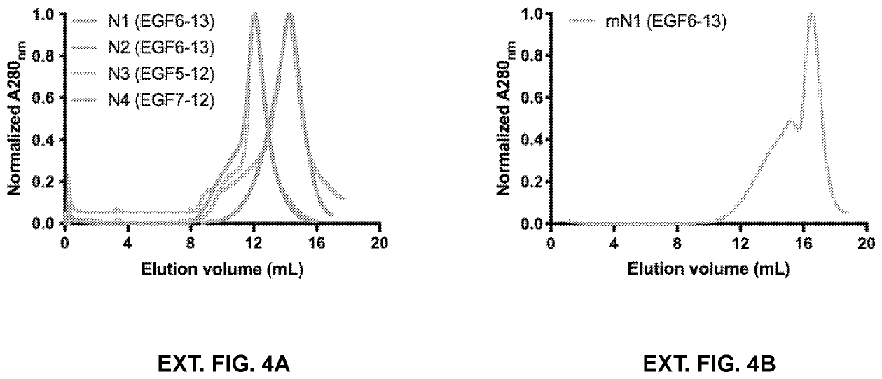

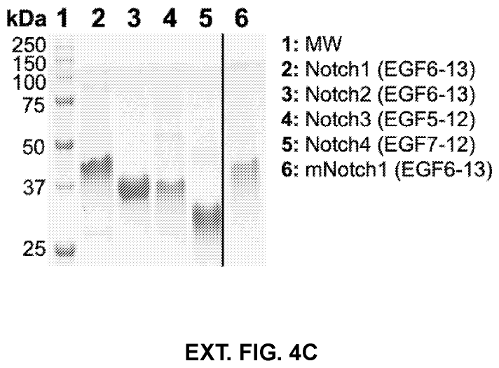

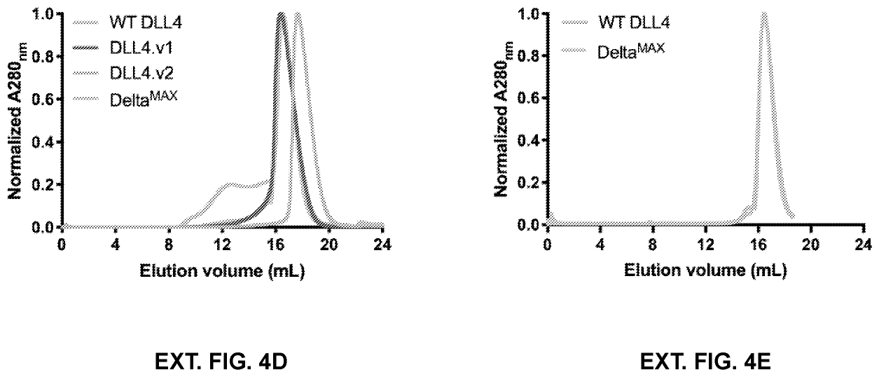

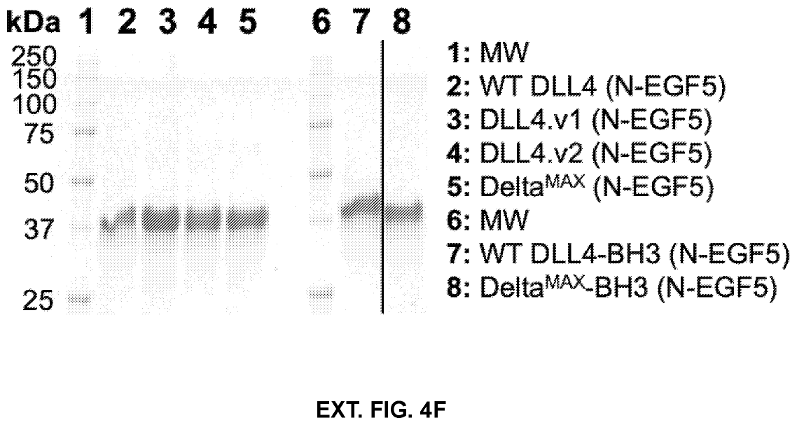

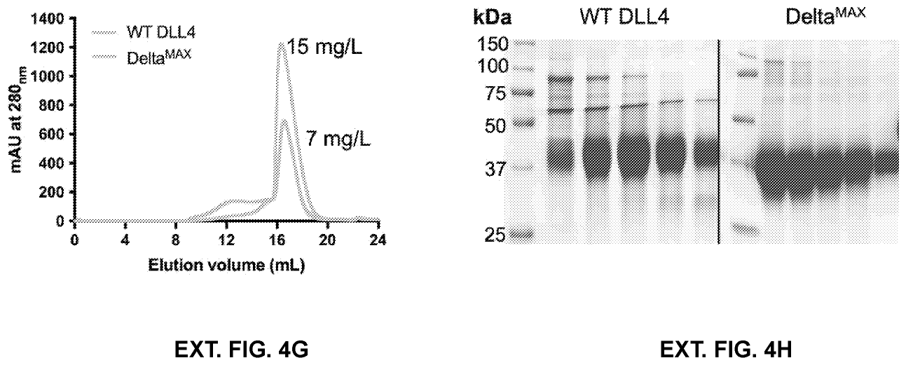

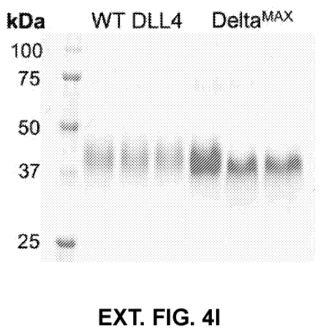

Extended Data FIG. 4. Purification of Notch and DLL4 proteins. (a-b) SEC chromatograms from the purification of the ligand-binding regions of human Notch1-4 (a) and murine Notch1 (b). All proteins were purified by Ni-NTA followed by size exclusion chromatography using a Superdex S75 column. c, SDS-PAGE gels showing the purity and molecular weight of each Notch construct. d, SEC chromatograms from the purification of DLL4, DLL4.v1, DLL4.v2, and Delta^{MAX} proteins. All proteins were purified by Ni-NTA followed by size exclusion chromatography using a Superdex S200 column. e, SEC chromatograms from the purification of C-termini biotinylated WT DLL4 and Delta^{MAX} proteins. f, SDS-PAGE gels showing the purity and molecular weight of each DLL4 construct. g, SEC chromatograms from 1 L protein preps were overlaid to highlight the increased yield of recombinant Delta^{MAX} compared to WT DLL4. h, SDS-PAGE gels showing the elution profile fractions of WT DLL4 and Delta^{MAX} preps from panel (g). i, Small scale preps of WT DLL4 and Delta^{MAX} performed in triplicate using the same baculovirus titers.

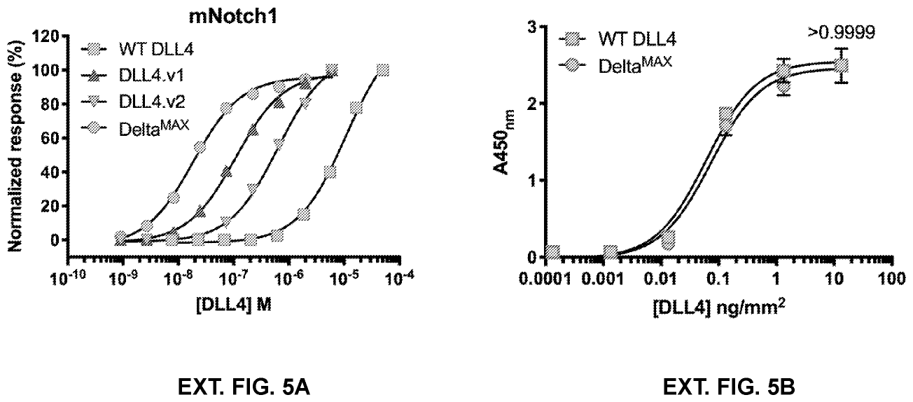

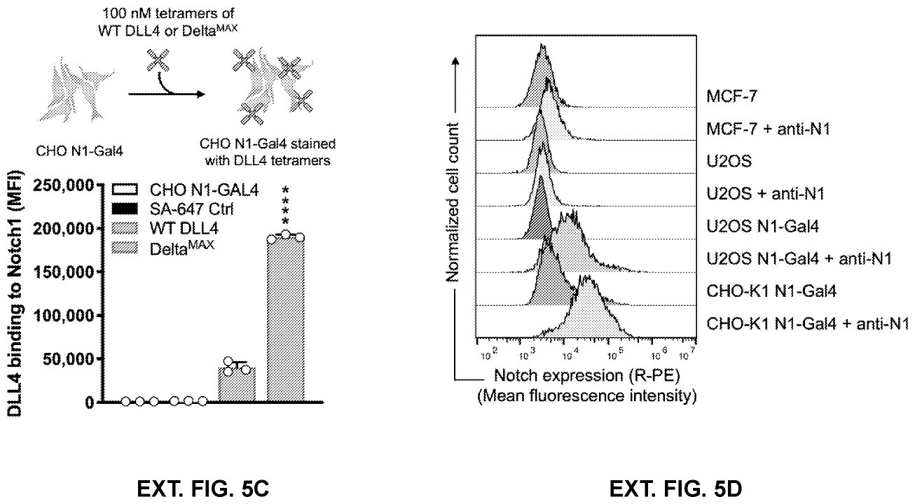

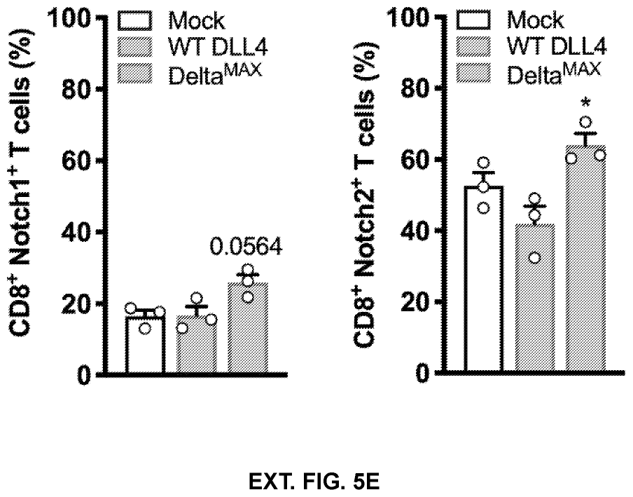

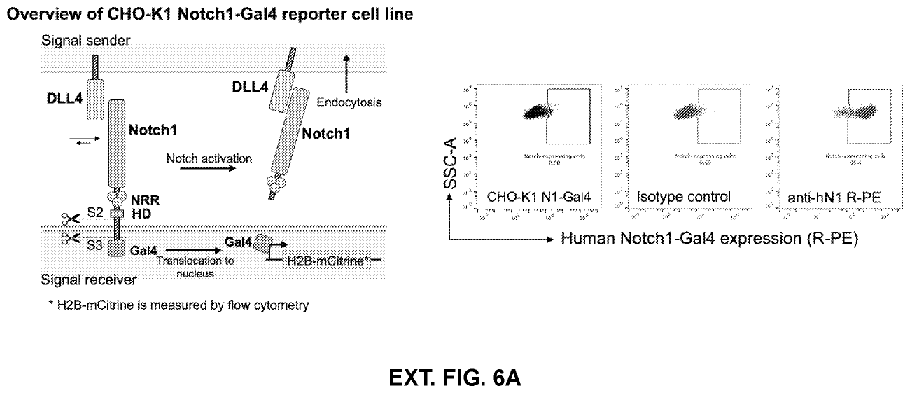

Extended Data FIG. 5. Quantitation of mNotch1 binding to DLL4 variants, ligand adsorption, and Notch surface expression. a, SPR binding isotherms were fitted to a 1:1 binding model to determine the binding affinity between mNotch1 (6-13) and each DLL4 variant. b, Determination of plate adsorption for WT DLL4 and Delta^{MAX} by ELISA. Data represent mean values±s.d. of three biological replicates. c, Binding assay of WT DLL4 and Delta^{MAX} tetramers to Notch1 overexpressing CHO-K1 N1-Gal4 cells was measured by flow cytometry. Data represent mean values±s.d. of three biological replicates. d, The level of Notch1 expression on the surface of cell lines used in this study was determined by flow cytometry. Data represent mean values±s.d. of three biological replicates. e, Percentage of CD8⁺ T cells expressing Notch1 (upper graph) and Notch2 (bottom graph) on cell surface determined by flow cytometry after stimulation with K32-WT DLL4, K32-Delta^{MAX}, or K32 for 96 hours. Data represent mean values±s.e.m. of three biological replicates. Delta^{MAX} statistics are referred to WT DLL4. *P<0.05, ****P<0.0001 (Two-way ANOVA).

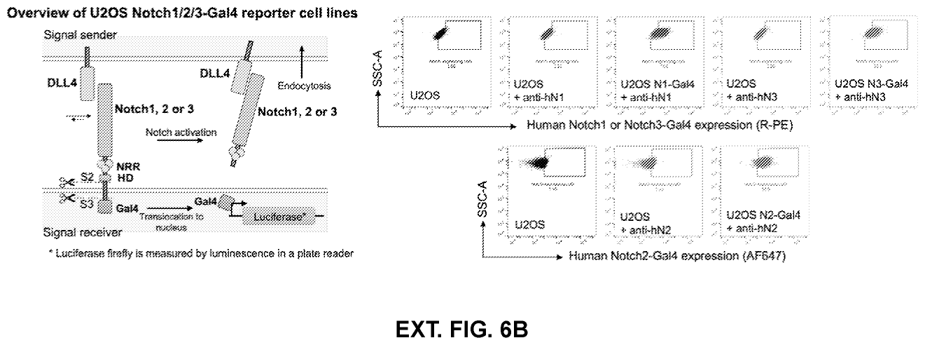

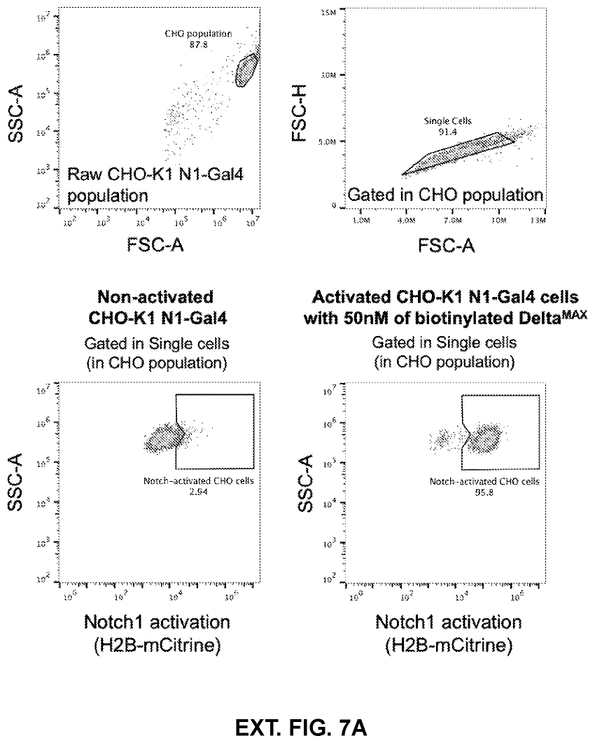

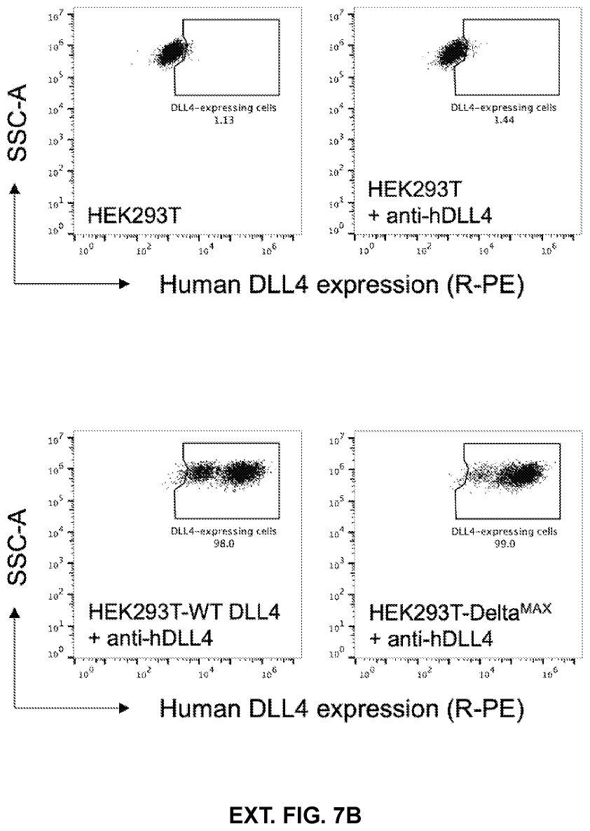

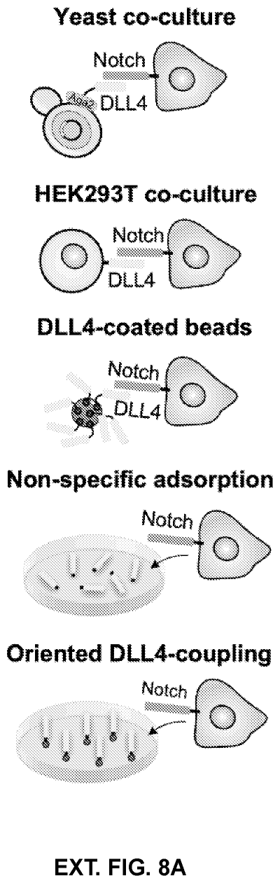

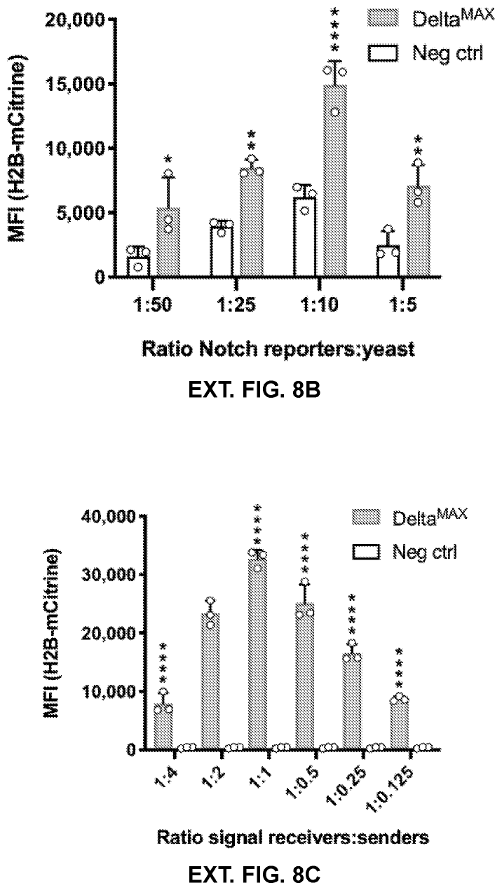

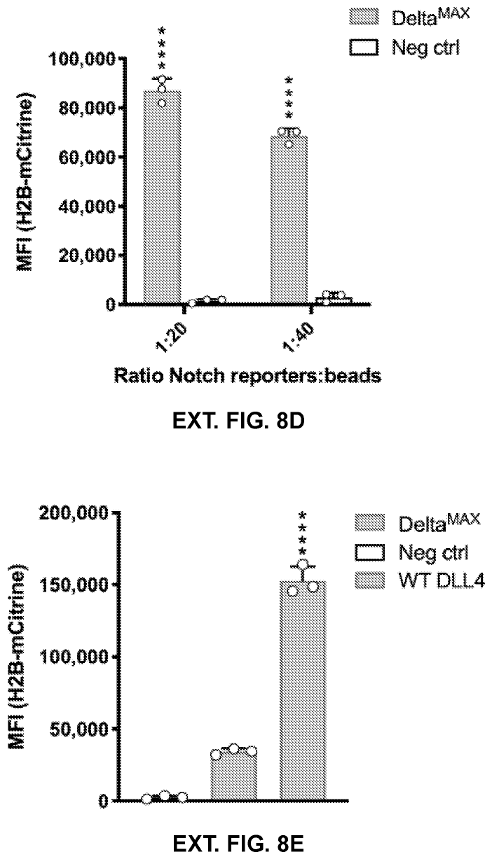

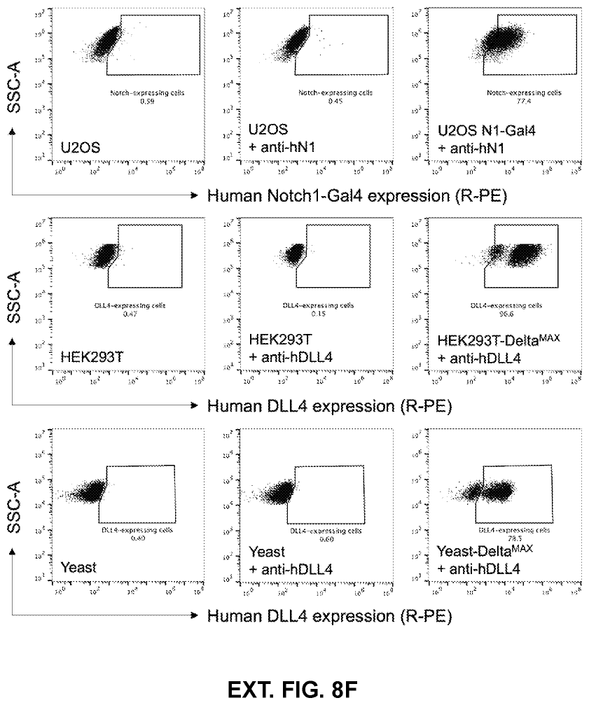

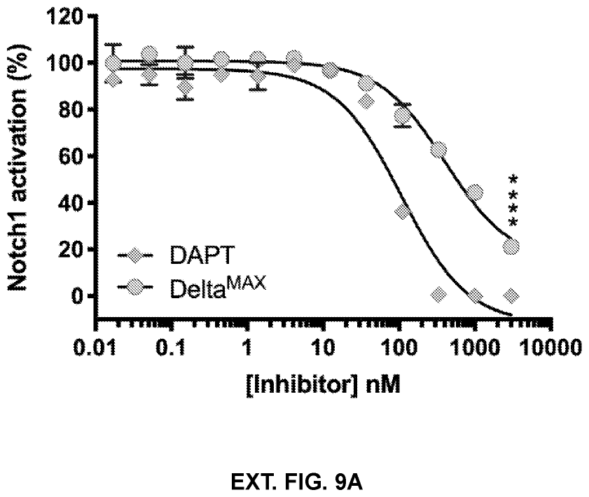

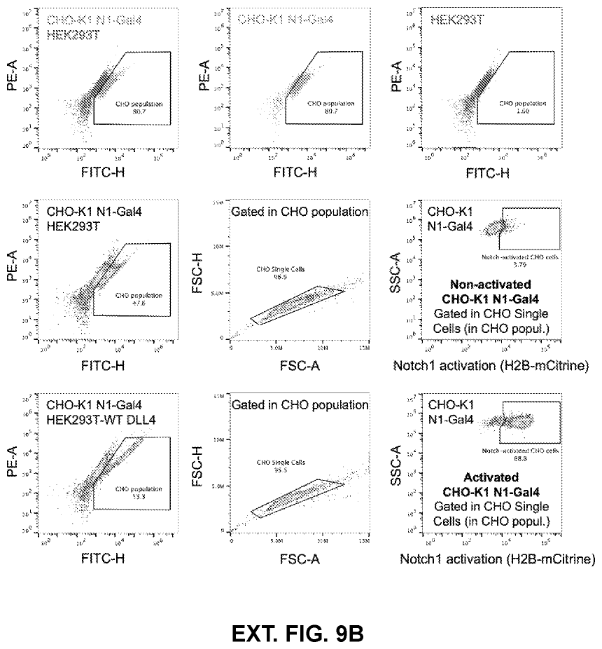

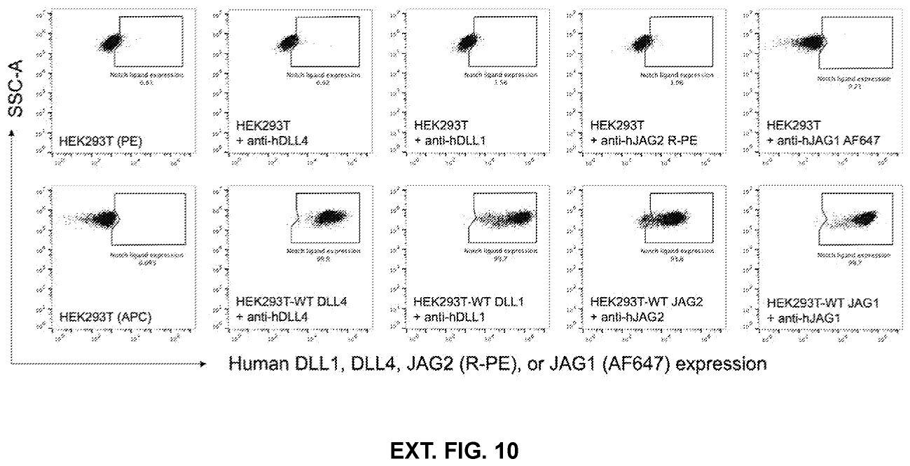

Extended Data FIG. 6. Fluorescent and luminescent Notch reporter cell lines. Cartoon schematics describing the fluorescent (a) and luminescent (b) Notch-Gal4 reporter systems used for signaling assays. Flow cytometry dot plots depict the staining of each cell line with Notch-specific antibodies to detect surface expression.

Extended Data FIG. 7. Gating strategy used in Notch co-culture assays. a, DLL4 variants were non-specifically adsorbed to 96-well tissue culture plates or immobilized to streptavidin plates. Next, Notch1 reporter cells (CHO-K1 N1-Gal4) were added to plates and Notch activation was measured by flow cytometry. The gating strategy to quantify Notch1 activation based on expression of H₂B-mCitrine is shown. HEK293T cells were transduced to generate stable cell lines expressing WT DLL4 or Delta^{MAX} and sorted to normalize the expression levels of each ligand. b, Expression of WT DLL4 and Delta^{MAX} on HEK293T cells was analyzed by flow cytometry following staining with anti-hDLL4 PE-conjugated antibody. The ligand-expressing cell lines were used for co-culture signaling assays with Notch1, Notch2, and Notch3-U2OS luciferase reporter cells. Density plots are representative of three biological replicates.

Extended Data FIG. 8. Optimization of different ligand presentation strategies. a, Cartoon representation of different ligand-presentation formats. b, Yeast expressing Delta^{MAX} (N-EGF5) were co-cultured with reporter cells for 24 h at different ratios and fluorescence was measured by flow cytometry. c, HEK293T cells expressing Delta^{MAX} were co-cultured with reporter cells at various ratios for 24 h and Notch activation was measured by flow cytometry. d, Magnetic beads were pre-coated with biotinylated Delta^{MAX} and co-cultured with reporter cells at ratios of 1:20 or 1:40 to stimulate Notch activation. e, SA-polystyrene beads were coated with biotinylated WT DLL4 or Delta^{MAX} and incubated in ratio 1:1 with CHO-K1 N1-Gal4 reporter cells to measure Notch activation by flow cytometry. f, Flow cytometry dot plots depict the expression level of Notch1 and Delta^{MAX}. Data in (b), (c), (d) and (e) represent mean values±s.d. of three biological replicates, while (f) are density plots representative of three biological replicates. Delta^{MAX} statistics are referred to WT DLL4. *P<0.05, **P<0.01, ****P<0.0001 (Two-way ANOVA).

Extended Data FIG. 9. Co-culture inhibition of Notch1 using DAPT or Delta^{MAX}. a, Dose-titration assay comparing the inhibition potency of DAPT and Delta^{MAX}. Fluorescent Notch1 reporter cells were cultured in a 1:1 ratio with HEK293T cells stably expressing WT DLL4. Data represent mean values±s.d. of three biological replicates. b, Summary of the gating strategy used for flow cytometry to differentiate between HEK293T and CHO-K1 N1-Gal4 cell signals. The basal expression of H₂B-mCitrine in CHO-K1 N1-Gal4 cells was used as a criterion to distinguish between cell types. Notably, the population identified in the middle and bottom panels (Notch1 reporter CHO-K1 N1-Gal4 cells) corresponds to approximately 50% of the total cells, which is consistent with a 1:1 ratio of CHO-K1 N1-Gal4 cells to 293T cells. Density plots are representative of three biological replicates. Delta^{MAX} statistics are referred to DAPT. ****P<0.0001 (Two-way ANOVA).

Extended Data FIG. 10. Expression of Notch ligands in stable cell lines. Stable HEK293T cell lines expressing WT DLL4, DLL1, JAG1, or JAG2 were stained with specific antibodies targeting the ECDs of each Notch ligand and measured by flow cytometry. The expression of Notch ligands, as well as sequencing results, validated these stable cell lines. Flow charts are representative of three biological replicates.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of chemistry, biology, and the like, which are within the skill of the art.

The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

It must be noted that, as used in the specification and the appended claims, the singular forms “a,” “an,” and “the” include plural referents unless the context clearly dictates otherwise.

Disclosed herein is a cell-free, bead-based system for generating cells of the T-cell lineage from stem or progenitor cells such as mouse or human hematopoietic stem/progenitor cells (HSPCs) or induced pluripotent stem cells (iPSCs). Non-plate-bound suspensions of engineered Notch ligands allow for the effective generation of T-lineage cells including progenitor T cells and mature T cells.

### Method for Generating Cells

Accordingly, the disclosure provides a method of generating cells of the T-cell lineage comprising (a) culturing a sample comprising stem cells or progenitor cells with a Notch ligand conjugated to a suspension support and (b) isolating cells of the T cell lineage.

The term “cells of the T cell lineage” refers to cells that show at least one phenotypic characteristic of a T cell or a precursor or progenitor thereof that distinguishes the cells from other lymphoid cells, and cells of the erythroid or myeloid lineages. Such phenotypic characteristics can include expression of one or more proteins specific for T-lineage on cells or a precursor or progenitor thereof, or a physiological, morphological, functional, or immunological feature specific for a T cell. Cells of the T cell lineage may be (a) progenitor or precursor cells committed to the T cell lineage (“progenitor T cells” or “proT cells”, as described herein); (b) CD25+ immature T cells; (c) cells that have undergone CD4 or CD8 lineage commitment (e.g. CD4+CD8lo TCRint cells); (d) characterized by TCR gene rearrangement; (e) precursor thymocytes that are CD4+CD8+ double positive (DP); (f) CD4−CD8+ or CD4+CD8− and optionally TCRhi; (g) CD3+CD90+, (h) single positive (SP) cells that are CD4−CD8+ or CD4+CD8− and TCRhi; (i) TCR-αβ+ and/or TCR-γδ+, (j) characterized by expression of any of multiple VB chains (e.g. Vβ-3, -6, and 17a); or (k) mature and functional or activated T cells which may be characterized as TCR/CD3hi, CD4−CD8+ or CD4+CD8−.

In one embodiment, a cell of the T cell lineage is a “progenitor T cell” or “proT cell”. The term “progenitor T cell” or “proT cell” as used herein means a T cell that is capable of maturing into a mature T cell or lymphocyte.

In one embodiment, the progenitor T cell is a human progenitor T cell. Phenotypes of human progenitor T cells include CD34+CD7+ and CD7+CD5+CD1a−. In another embodiment, the progenitor T cell is a mouse progenitor T cell. Phenotypes of mouse progenitor T cells include CD25+.

In another embodiment, a cell of the T cell lineage is a CD4 and CD8 double positive (DP) cell characterized by CD4+CD8+ or CD4+CD8+CD3+ phenotype. In another embodiment, a cell of the T cell lineage is a CD4 or CD8 single positive (SP) cell characterized by CD4−CD8+, CD4+CD8− or CD4−CD8+CD3+, CD4+CD8−CD3+.

The term “suspension support” as used herein, refers to any material that when conjugated to a Notch ligand or other T cell co-stimulatory molecule, allows the Notch ligand (or co-stimulatory molecule) to be suspended in culture media. The suspension support can be made from a wide variety of materials and can be in a variety of formats. Examples of supports that can be used as suspension supports include, but are not limited to, particles, beads (including microbeads), proteins, lipids, nucleic acid molecules, filters, fibers, screens, mesh, tubes, hollow fibers, biological tissues and any combinations thereof.

In one embodiment, the suspension support is a particle. The particular may be of any shape, including but not limited to a sphere, oval, rod, or rectangle. The particle can be of a variety of materials, including, but not limited to natural or synthetic polymers, natural or synthetic waxes, ceramics, metals, biological materials or combinations thereof.

In one embodiment, the suspension support is a microbead. The term “microbead” or “ubead” as used herein refers to a spherical or roughly spherical bead having a diameter from 0.01 μm (10 nm) to 500 μm, optionally from 1 to 200 μm. In another embodiment, the microbead has a diameter of 6.5 to 100 μm, optionally 20 to 30 μm, 24 to 26 μm or 25 μm.

Various types of microbeads are contemplated herein. In one embodiment, the microbead is a polymer, silica or magnetic microbead. In other embodiments, the microbead is a polystyrene microbead, a gold nanoparticle or a Dynabead. In another embodiment, the microbead is a co-polymer of lactic and glycolic acid (PLGA).

Various means of conjugating proteins to supports are known in the art. A protein may be directly or indirectly conjugated to a suspension support, for example a microbead.

In one embodiment, the Notch ligand described herein is conjugated to a suspension support using a biotin/streptavidin system. Here, the Notch ligand is biotinylated and then conjugated to streptavidin-coated suspension support (for example, a streptavidin-coated microbead). In another embodiment, the Notch ligand described herein is conjugated to a suspension support via protein-G or protein A.

The term “Notch ligand” as used herein refers to a ligand capable of binding to a Notch receptor polypeptide present in the membrane of a number of different mammalian cells including hematopoietic stem/progenitor cells. The Notch receptors that have been identified in human cells include Notch-1, Notch-2, Notch-3 and Notch-4. Notch ligands typically have a diagnostic DSL domain (D-Delta, S-Serrate, and L-Lag2) comprising 20-22 amino acids at the amino terminus and between 3 to 8 EGF repeats on the extracellular surface.

A Notch ligand is selected that promotes and maintains differentiation and proliferation of cells of the T cell lineage. The Notch ligand is optionally human in origin, or may be derived from other species, including mammalian species such as rodent, dog, cat, pig, sheep, cow, goat and primates.

Particular examples of Notch ligands include the Delta family. The Delta family includes Delta-1 (Genbank Accession No. AF003522, Homo sapiens), Delta-3 (Genbank Accession No. AF084576, Rattus norvegicus), Delta-like 1 (DL1, Genbank Accession No. NM_005618 and NP_005609, Homo sapiens; Genbank Accession No. X80903, 148324, M. musculus), Delta-like 3 (Genbank Accession No. NM_053666, N_446118, Rattus norvegicus), Delta-4 (Genbank Accession No. AF273454, BAB18580, Mus musculus; Genbank Accession No. AF279305, AAF81912, Homo sapiens), and Delta-like 4 (DL4; Genbank Accession. No. Q9NR61, AAF76427, AF253468, NM_019074, Homo sapiens; Genbank Accession No. NM 019454, Mus musculus). Notch ligands are commercially available or can be produced by recombinant DNA techniques and purified to various degrees.

Optionally, the Notch ligand comprises at least one protein tag. A protein tag is a peptide sequence appended to a protein of interest such as a Notch ligand. The protein tag may be directly or indirectly linked to the protein of interest. Various protein tags are known in the art and can be used for a number of purposes. In one embodiment, the Notch ligand comprises an Fc tag (also known as an Fc-fusion protein). As used herein, the term “Fc” refers to the Fc domain of IgG. In one particular embodiment, Notch ligand DL4 is fused to Fc (DL4-Fc). In another embodiment, the tag is a His tag. In a further embodiment, the tag is a molecule that facilitates oligomerization of the Notch ligand. For example, a small domain of COMP (cartilage oligomeric matrix protein) can be fused to the Notch ligand (for example, DL4) to form DL4 pentamers. Ferritin can be used in a similar manner to form DL4 multimers.

According to the methods described here, cells of the T cell lineage are generated by culturing a sample comprising stem cells or progenitor cells. Stem or progenitor cells may be obtained from any suitable source, including, without limitation, umbilical cord blood, embryos, embryonic tissue, fetal tissue, bone marrow and blood. In one embodiment, the stem or progenitor cell is a hematopoietic stem or progenitor cell (HSPC). In another embodiment, the stem cell is an embryonic stem cell (ESC). In a further embodiment, the stem or progenitor cell is an induced pluripotent stem cell. In another embodiment, the stem or progenitor cell is a CD34+ hematopoietic precursor cell, optionally a CD34+ hemogenic endothelial precursor cell that has been differentiated from an ESC or iPSC, or a CD34+ pre-hematopoietic cell differentiated from an ESC or pluripotent stem cell (PSC). Various differentiation protocols for obtaining CD34+ cells are known in the art. For therapeutic applications, the stem cells or progenitor cells used to generate the cells of the T cell lineage may be obtained from the patient to be treated.

The term “hematopoietic stem/progenitor cell”, “hematopoietic stem or progenitor cell” or “HSPC” as used herein refers to undifferentiated hematopoietic cells that are capable of differentiation to other cell types, including cells of the T cell lineage. HSPCs can be obtained from a number of sources including, but not limited to bone marrow, umbilical cord blood and mobilized peripheral blood (mPB). HSPCs can also be obtained from several fetal and embryonic sites, such as liver, yolk sac or dorsal aorta. HSPCs can also be obtained by inducing the differentiation of ESCs or iPSCs in culture.

The term “embryonic stem cell” or “ESC” as used herein refers to undifferentiated embryonic stem cells that have the ability to integrate into and become part of the germ line of a developing embryo.

The term “induced pluripotent stem cell” or “iPSC” as used herein refers to cells derived from somatic cells, such as skin or blood cells that have been reprogrammed back into an embryonic-like pluripotent state. In one embodiment, iPSCs are derived from T cells with a known or unknown TCR specificity (for example, T cells bearing TCRs with specificity against cancer).

Typically, a sample containing stem or progenitor cells is first depleted of non-stem cells or mature cells. Negative and positive selection methods known in the art may be used for enrichment of the stem or progenitor cells. For example, cells can be sorted based on cell surface antigens using a fluorescence activated cell sorter, or magnetic beads which bind cells with certain cell surface antigens. Negative selection columns can be used to remove cells expressing lineage specific surface antigens.

In an embodiment, a sample containing stem or progenitor cells is separated into lineage-negative (Lin−) and lineage position (Lin+) fractions. The Lin-fraction can be sorted for CD34+ cells.

The progenitor cells or stem cells are cultured under suitable conditions as described herein to generate cells of the T cell lineage. Preferably, the cells are cultured in the presence of one or more Notch ligands conjugated to a suspension support for a sufficient time to form cells of the T cell lineage.

One advantage of the methods described herein is that they allow the cells of the T cell lineage to be cultured in suspension. In an embodiment, the progenitor cells or stem cells are cultured in suspension with a Notch Ligand conjugated to a suspension support such as a microbead. In a suspension culture, cells grow free-floating in a culture medium. In contrast, in an adherent culture, cells grow as monolayers on an artificial substrate.

In another embodiment, the progenitor cells or stem cells are cultured in suspension in a bioreactor, optionally a closed or a closed, automated bioreactor, with a Notch ligand conjugated to a suspension support. In one embodiment, the suspension support is a microbead that has a diameter that is compatible with the bioreactor. Various bioreactors are known in the art and can include batch, fed batch or continuous bioreactors. An example of a continuous bioreactor is a continuous stirred-tank reactor model.

Various concentrations of progenitor cells or stem cells in the culture are contemplated. For example, the concentration of progenitor cells or stem cells in the culture may be anywhere from 1 to millions of cells per ml of media.

In one embodiment, the ratio of microbead-conjugated Notch ligand to progenitor cells or stem cells is between 1:1 and 27:1, optionally 5:1 to 15:1, 8:1 to 10:1 or 9:1. This ratio is also referred to herein as the “microbead to cell ratio”.

The inventors have also shown that the direction of the orientation of the Notch ligand to the suspension support can enhance Notch signaling. Accordingly, in one embodiment, the C-terminus of the Notch ligand is conjugated to the suspension support. This can be engineered, for example, by adding a sequence at the C-terminal end of the Notch ligand that can be enzymatically conjugated to a biotin molecule. In another embodiment, the Fc segment of the fusion protein, Notch ligand-Fc, present in the C-terminal region, can directly bind to protein A or protein G that is conjugated to the suspension support.

One or more positive cytokines that promote commitment and differentiation of cells of the T cell lineage may also be added to the culture. The cytokines may be human in origin, or may be derived from other species. The concentration of a cytokine in a culture is typically about 1-10 ng/ml. The following are representative examples of cytokines that may be employed in the present application: all members of the Flt-3-ligand, and interleukin-7 (IL-7) and Stem Cell Factor. In one embodiment, the cytokines used herein are Flt-3-ligand and IL-7 and Stem Cell Factor. The cytokines may be used in combination with equal molar or greater amounts of a glycosaminoglycan such as heparin sulfate. The cytokines are commercially available or can be produced by recombinant DNA techniques and purified to various degrees. Some of the cytokines may be purified from culture media of cell lines by standard biochemical techniques.

One or more additional molecules, each conjugated to a suspension support, may also be added to the culture. In one embodiment, the additional molecule is a molecule that promotes T cell development (for example, promotes commitment and differentiation of cells of T cell lineage), also referred to herein as a “T cell co-stimulatory molecule”. In one example, the inventors have shown that microbead-conjugated DL4 and VCAM1 cultured with HSPCs accelerated differentiation to the T cell lineage. Thus, in one embodiment, the T cell co-stimulatory molecule is VCAM1. As used herein, the term “VCAM1” refers to Vascular cell adhesion protein 1 also known as vascular cell adhesion molecule 1 (VCAM1) or cluster of differentiation 106 (CD106), a protein that in humans is encoded by the VCAM1 gene. The term “VCAM1” also includes a mutant or variant of a VCAM1. In another embodiment, the T cell co-stimulatory molecule is a cytokine or chemokine (Stem Cell Factor, IL-7, CCL25, or CXCR4), Major Histocompatibility Complex (MHC) class I or class II, or co-stimulatory (CD80, CD86) molecule. Optionally, the T cell co-stimulatory molecule comprises at least one protein tag. Various protein tags are known in the art and can be used for a number of purposes. In one embodiment, the T cell co-stimulatory molecule comprises an Fc tag (also known as an Fc-fusion protein).

The progenitor cells and stem cells may be cultured in culture media comprising conditioned media, non-conditioned media, or embryonic stem cell media. Examples of suitable conditioned media include IMDM, DMEM, or αMEM, conditioned with embryonic fibroblast cells (e.g. human embryonic fibroblast cells or mouse embryonic fibroblast cells), or equivalent media. Examples of suitable non-conditioned media include Iscove's Modified Dulbecco's Medium (IMDM), DMEM, or αMEM, or equivalent media. The culture media may comprise serum (e.g. bovine serum, fetal bovine serum, calf bovine serum, horse serum, human serum, or an artificial serum substitute) or it may be serum free. Other examples of media useful in the present methods include StemCell Technologies media (StemSpan™ SFEM II) or any other commercially available equivalent media.

In one embodiment, the culture conditions entail culturing the progenitor cells or stem cells for a sufficient period of time so that cells in the preparation form proT cells. In another embodiment, the culture conditions entail culturing the progenitor cells or stem cells for a sufficient period of time so that cells in the preparation form mature T cells, for example mature SP T cells. It will be appreciated that the cells may be maintained for the appropriate amount of time required to achieve the desired cellular composition. Optionally, the progenitor cells or stem cells are cultured for at least 6, 8, 10, 12, 14, 21, 28, 35 or 42 days. In one example, the progenitor cells or stem cells are cultured with the Notch ligand described herein for 4 to 21 days, 6 to 18 days or 7 to 14 days to generate proT cells. In another example, the progenitor cells or stem cells are cultured with the Notch ligand described herein for at least 21, 28, 35 or 42 days to generate mature T cells.

The methods of the present application allow the generation of large numbers of cells of the T cell lineage. In particular, in one embodiment, following 14 or more days of culture, greater than 50-fold, 75-fold, 100-fold, 125-fold, 150-fold, 175-fold or 200-fold cell expansion over the initial starting number of stem cells or progenitor cells is obtained.

The term “isolated” as used herein means that the progenitor cell has been separated or purified from cellular or biological material found with the cells in their native environment. It thus distinguishes the cells from how they exist in nature.

The term “a cell” or “the cell” includes a plurality of cells.

### Engineered Broadly Reactive Human Notch Ligands

Disclosed herein is an engineered Notch ligand that involves an engineered DLL4 proteins having a conservative amino acid substitution at a residue corresponding to residues 28, 107, 143, 194, and 206 as set forth in SEQ ID NO: 1 or SEQ ID NO: 7 and/or comprising or further comprising at least one conservative amino acid substitution at residues 256, 257, 271, 280, 301, and 305 as set forth in SEQ ID NO: 1 or SEQ ID NO: 7. For example, the substitution of the engineered DLL4 protein can comprise a glycine to serine substitution at residue 28 (G28S) (as in SEQ ID NOs: 2, 3, 4, 10, 11, and 12), a phenylalanine to leucine substitution at residue 107 (F107L) (as in SEQ ID NOs: 2, 3, 4, 10, 11, and 12), an isoleucine to phenylalanine substitution at residue 143 (I143F) (as in SEQ ID NOs: 2, 3, 4, 10, 11, and 12), a histidine to tyrosine substitution at residue 194 (H194Y) (as in SEQ ID NOs: 2, 3, 4, 10, 11, and 12), and a leucine to proline substitution at residue 206 (L206P) (as in SEQ ID NOs: 2, 3, 4, 10, 11, and 12).

In some embodiments, the engineered DLL4 proteins can comprise one, two, three, four, five, or six substitutions at residues 256, 257, 271, 280, 301, and 305 as set forth in SEQ ID NO: 1 or SEQ ID NO: 7.

Thus, it is understood and herein contemplated that any one, or combination of any of the residues 256, 257, 271, 280, 301, and 305 can comprise a native residue or substitution. Accordingly, in one aspect, disclosed herein are engineered DLL4 proteins wherein the amino acid at residue 256 comprises a histidine, tyrosine, phenylalanine, leucine, asparagine, isoleucine, valine, or aspartic acid. For example, the amino acid at residue 256 can comprise a histidine or a substitution from histidine in wild-type (WT) human DLL4 (as set forth in SEQ ID NO: 1 or 7) to a tyrosine (a H256Y substitution), to a phenylalanine (a H256F substitution), a leucine (a H256L substitution), an asparagine (a H256N substitution), a isoleucine (a H256I substitution), a valine (a H256V substitution), or aspartic acid (a H256D substitution) as set forth in SEQ ID NOs: 3, 6, 9, and 12. Similarly, the engineered DLL4 proteins can comprise a proline, histidine, leucine, isoleucine, threonine, asparagine, tyrosine, serine, or phenylalanine at residue 257. Thus, in one aspect, the engineered DLL4 protein can comprise an asparagine at residue 257 or substitution from the asparagine in wild-type (WT) human DLL4 (as set forth in SEQ ID NO: 1 or 7) to a tyrosine (a H257Y substitution), a proline (a N257P substitution), a histidine (a N257H substitution), a leucine (a N257L substitution), an isoleucine (a N257I substitution), a threonine (a N257T substitution), a serine (a N257S substitution), or a phenylalanine (a N257F substitution) as set forth in SEQ ID NOs: 3, 4, 5, 6, 9, 10, 11, and 12. In one aspect, disclosed herein are engineered DLL4 proteins wherein the amino acid at residue 271 comprises a leucine, proline, histidine, asparagine, threonine, or isoleucine (such as, for example, a wild-type residue as set forth in SEQ ID NO: 1 or 7 (i.e., the threonine) or a substitution of the threonine for a leucine (a T271L substitution), a threonine to proline substitution (a T271P substitution), a threonine to histidine substitution (a T271H substitution), a threonine to arginine substitution (a T271N substitution), or a threonine to isoleucine substitution (a T271I substitution) as set forth in SEQ ID NOs: 3, 4, 5, 6, 9, 10, 11, and 12. Also disclosed herein are engineered DLL4 proteins, wherein the amino acid at residue 280 comprises a phenylalanine or a substitution of the phenylalanine with a leucine (a F280L substitution), a tyrosine (a F280Y substitution), or histidine (a F280H substitution) as set forth in SEQ ID NOs: 3, 4, 5, 6, 9, 10, 11, and 12. Additionally, in one aspect, the disclosed engineered DLL4 proteins can comprise the native serine amino acid at residue 301 as set forth in SEQ ID NO: 1 or 7, or comprise a substitution of the serine for an asparagine (a S301N substitution), arginine (a S301R substitution) as set forth in SEQ ID NO: 4, 5, 10, or 11, or a histidine (a S301H substitution) as set forth in SEQ ID NO: 4, 6, 10, or 12. Also disclosed herein are engineered DLL4 proteins, wherein the amino acid at residue 305 comprises a glutamine, proline, arginine, or leucine and thus can comprise the wild-type amino acid as set forth in SEQ ID NO: 1 (i.e., a glutamine) or a substitution of the glutamine for a proline (a Q305P substitution), a substitution of the glutamine for an arginine (a Q305R substitution), or a substitution of the glutamine for a leucine (a Q305L substitution) as set forth in SEQ ID NOs: 3, 4, 5, 6, 9, 10, 11, and 12. In one aspect, disclosed herein are engineered DLL4 proteins of any preceding aspect, wherein the DLL4 protein comprises SEQ ID NOs: 2, 3, 4, 5, 6, 8, 9, 10, 11, and 12.

### Suspension Notch Ligands

Also disclosed herein is a suspension of the disclosed engineered Notch ligands. As used herein, the term “suspension Notch ligand” refers to an engineered Notch ligand disclosed herein for use in a suspension cell culture.

Accordingly, the disclosure also provides a suspension Notch ligand as described herein. The suspension Notch ligand comprises (a) engineered Notch ligand disclosed herein and (b) a suspension support, wherein the engineered Notch ligand conjugated to the suspension support.

As disclosed herein, the disclosed engineered Notch ligands when directly conjugated to microbeads deliver a strong and sustained signal to induce HSPCs to develop into cells of the T cell lineage.

In some embodiments, the direction of the orientation of the Notch ligand to the suspension support can enhance Notch signaling. Accordingly, in some embodiments, the C-terminus of the Notch ligand is conjugated to the suspension support. This can be engineered, for example, by adding a sequence at the C-terminal end of the Notch ligand that can be enzymatically conjugated to a biotin molecule.

### Kits

Suspension Notch ligands may be prepared and packaged in kits for use in generating cells of the T cell lineage.

Accordingly, also provided herein is a kit for producing cells of the T cell lineage comprising a suspension Notch ligand, wherein the suspension Notch ligand comprises (a) a Notch ligand and (b) a suspension support, wherein the Notch ligand is conjugated to the suspension support. Optionally, the suspension Notch ligand is contained in a preservative and/or buffer solution and the kit further comprises a device for dispensing the suspension Notch ligand such as a vial or syringe.

In one embodiment, the kit further comprises culture media for culturing a sample comprising stem cells or progenitor cells with a suspension Notch ligand. Examples of culture media include conditioned media, non-conditioned media, or embryonic stem cell media. The culture media may comprise serum (e.g. bovine serum, fetal bovine serum, calf bovine serum, horse serum, human serum, or an artificial serum substitute) or it may be serum free. Other examples of culture media useful include StemCell media or any other commercially available equivalent media.

In another embodiment, the kit further comprises one or more additional molecules, each conjugated to a suspension support. In one embodiment, the additional molecule is a molecule that promotes T cell development (for example, promotes commitment and differentiation of cells of T cell lineage), also referred to herein as a “T cell co-stimulatory molecule”. In another embodiment, the T cell co-stimulatory molecule is VCAM1.

The media optionally includes one or more cytokines that promote commitment and differentiation of cells of the T cell lineage. The cytokines may be human in origin, or may be derived from other species. The concentration of a cytokine in a culture is typically about 1-10 ng/ml. The following are representative examples of cytokines that may be employed in the present application: all members of the Flt-3-ligand, and interleukin-7 (IL-7) and Stem Cell Factor. In one embodiment, the cytokines used herein are Flt-3-ligand and IL-7 and Stem Cell Factor. The cytokines may be used in combination with equal molar or greater amounts of a glycosaminoglycan such as heparin sulfate. The cytokines are commercially available or can be produced by recombinant DNA techniques and purified to various degrees. Some of the cytokines may be purified from culture media of cell lines by standard biochemical techniques.

In one embodiment, the kit comprises one or more containers for the within-described reagents.

Printed instructions providing guidance in the use of the reagent(s) may also be included in the kit, in various embodiments. The term “instructions” or “instructions for use” typically includes a tangible expression describing the reagent concentration or amount of the suspension Notch ligand and/or at least one assay method parameter, such as the relative amounts of suspension Notch ligand and cells to be mixed, culturing time periods, temperature, media conditions, and the like. For example, in one embodiment, the instructions describe a method comprising (a) culturing a sample comprising stem cells or progenitor cells with a Notch ligand conjugated to a suspension support and (b) isolating cells of the T cell lineage.

### Cells of the T Cell Lineage

The disclosure further provides cells of the T cell lineage generated by the methods, systems and kits described herein, or mitotic or differentiated cells that are progeny of the cells.

In one embodiment, the disclosure provides a “progenitor T cell” or “proT cell” generated by the methods described herein. In another embodiment, the progenitor T cell is a human progenitor T cell, for example a human progenitor T cell characterized by CD34+CD7+ or CD7+CD5+CD1a−.

In another embodiment, the progenitor T cell is a mouse progenitor T cell, for example a mouse progenitor T cell characterized by CD25+.

The disclosure also provides a double positive (DP) T-cell characterized by CD4+CD8+ or CD4+CD8+CD3+. The disclosure further provides a cell of the T-cell lineage that is a single positive (SP) cell characterized by CD4−CD8+, CD4+CD8− or CD8+CD3+, CD4+CD3+.

In one embodiment, a cell of the T cell lineage generated by the methods described herein (for example, a progenitor T cell or a mature T cell) is engineered with a T cell receptor (TCR) or a chimeric antigen receptor (CAR) to confer specificity to tumor associated antigens (TAA). Cells engineered as such can be useful for treating conditions such as cancer.

In another aspect, the present disclosure provides a pharmaceutical composition comprising isolated cells of the T cell lineage generated by the methods described herein and a pharmaceutically acceptable diluent or carrier.

Suitable diluents and carriers are described, for example, in Remington's Pharmaceutical Sciences. On this basis, the compositions include, albeit not exclusively, solutions of the proT cells in association with one or more pharmaceutically acceptable vehicles or diluents, and contained in buffered solutions with a suitable pH and iso-osmotic with physiological fluids.

Pharmaceutical compositions include, without limitation, lyophilized powders or aqueous or non-aqueous sterile injectable solutions or suspensions, which may further contain antioxidants, buffers, bacteriostats and solutes that render the compositions substantially compatible with the tissues or the blood of an intended recipient. Other components that may be present in such compositions include water, surfactants (such as Tween™), alcohols, polyols, glycerin and vegetable oils, for example. Extemporaneous injection solutions and suspensions may be prepared from sterile powders, granules, tablets, or concentrated solutions or suspensions. The composition may be supplied, for example but not by way of limitation, as a lyophilized powder which is reconstituted with sterile water or saline prior to administration to the patient.

Pharmaceutical compositions also include cyropreservative solutions. In one embodiment, cells of the T cell lineage generated by the methods described herein are cryopreserved in appropriate media, for example pharmaceutically acceptable or GMP-grade media and optionally formulated for administration to a subject in need thereof.

Suitable pharmaceutically acceptable carriers include essentially chemically inert and nontoxic compositions that do not interfere with the effectiveness of the biological activity of the pharmaceutical composition. Examples of suitable pharmaceutical carriers include, but are not limited to, water, saline solutions, glycerol solutions, ethanol, N-(1 (2,3-dioleyloxy) propyl) N,N, N-trimethylammonium chloride (DOTMA), diolesyl-phosphotidyl-ethanolamine (DOPE), and liposomes. Such compositions should contain a therapeutically effective amount of the compound, together with a suitable amount of carrier so as to provide the form for direct administration to the patient.

The compositions can be administered for example, by parenteral, intravenous, subcutaneous, intramuscular, intracranial, intraorbital, ophthalmic, intraventricular, intracapsular, intraspinal, intracisternal, intraperitoneal, intranasal, aerosol or oral administration. For parenteral administration, solutions of the pro-T cells described herein can be prepared in water suitably mixed with a surfactant such as hydroxypropylcellulose. Dispersions can also be prepared in glycerol, liquid polyethylene glycols, DMSO and mixtures thereof with or without alcohol, and in oils. Under ordinary conditions of storage and use, these preparations contain a preservative to prevent the growth of microorganisms. A person skilled in the art would know how to prepare suitable formulations.

Preferably the cells of the T cell lineage are present in an amount effective for treating a disease state in a subject need thereof. In one embodiment the cell of the T cell lineage is present in an amount effective to enhance hematopoietic progenitor cell engraftment in a subject in need thereof. Optionally, the composition further comprises cells of the T cell lineage, or tissue for transplantation. In one embodiment the tissue comprises a thymus. In another embodiment the tissue comprises an organ.

### Therapeutic Applications

The ability to generate in vitro-derived human progenitor T cells and to test their safety in human/mouse immune engraftment models, opens avenues for cellular based approaches for treating immune-related disorders of the T lineage (Legrand et al., 2006; van den Brink et al., 2004). T cells are the major effector arm of the adaptive immune system in recognizing and eliminating viral and bacterial pathogens. In certain rare blood cancers, such as T cell acute lymphoblastic leukemia (T-ALL), T cells proliferate, crowding out healthy immune cells and perturbing normal immune function (Ferrando et al., 2002; Weng et al., 2004). Although chemotherapy can often impart therapeutic benefits in cancer patients, it often can lead to immuno-deficiency and susceptibility to opportunistic infections. Opportunistic infections also pose a serious concern in AIDS patients whose CD4+ T cells have been depleted following infection with HIV. While immunodeficiency remains a serious concern in HIV/AIDS and cancer, immune-hyperactivity is equally problematic in autoimmune disease where T cells that lack proper regulatory control generate immune responses to self-tissue.

Accordingly, the present application includes a method of treating a subject having a condition requiring an increase in the number of T cells comprising:
(i) generating cells of the T cell lineage comprising (a) culturing a sample comprising stem cells or progenitor cells with an engineered Notch ligand disclosed herein conjugated to a suspension support, optionally a particle or a microbead, and (b) isolating cells of the T cell lineage, and
(ii) administering an effective amount of the cells of the T cell lineage to a subject in need thereof.

In one embodiment, the cells of the T cell lineage are progenitor T cells.

In another embodiment, the cells of the T cell lineage are mature T cells.

The disclosure also provides a use of cells of the T cell lineage, optionally progenitor T cells or mature T cells, generated by the methods described herein for treating a subject having a condition requiring an increase in the number of T cells.

The disclosure also provides a use of cells of the T cell lineage, optionally progenitor T cells or mature T cells, generated by the methods described herein for use in regenerative medicine, for example to replace and/or regenerate tissues affected by disease or trauma.

As used herein, the phrase “effective amount” or “therapeutically effective amount” means an amount effective, at dosages and for periods of time necessary to achieve the desired result. Effective amounts may vary according to factors such as the disease state, age, sex, weight of the subject. The amount of a given cell preparation that will correspond to such an amount will vary depending upon various factors. Such as the pharmaceutical formulation, the route of administration, the type of disease or disorder, the identity of the subject or host being treated, and the like, but can nevertheless be routinely determined by one skilled in the art. An “effective amount” will preferably be an amount effective for the cell of the T cell lineage to engraft the subject being treated.

The term “treating” or “treatment” as used herein and as is well understood in the art, means an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, diminishment of the reoccurrence of disease, and remission (whether partial or total), whether detectable or undetectable. “Treating” and “treatment” can also mean prolonging survival as compared to expected survival if not receiving treatment. “Treating” and “treatment” as used herein also include prophylactic treatment.

The term “subject” as used herein means any member of the animal kingdom and is preferably a human.

A “condition requiring an increase in number of T cells” includes any condition wherein T cell levels are reduced as compared to a healthy animal, including, without limitation, immunodeficiency, cancer, genetic diseases (for example, Primary Immunodeficiency Diseases (PIDs)), infectious diseases, immune disorders and autoimmunity.

As set out above, the cells of the T-cell lineage described herein can be engineered to express T cell receptors (TCRs) or a chimeric antigen receptors (CARs) that specifically recognize tumor associated antigens.

Accordingly, the present application also includes a method of treating cancer in a subject comprising:
(i) generating cells of the T cell lineage comprising (a) culturing a sample comprising stem cells or progenitor cells with a Notch ligand conjugated to a suspension support, optionally a particle or a microbead, and (b) isolating cells of the T cell lineage, and
(ii) administering an effective amount of the cells of the T cell lineage to a subject in need thereof, wherein the cells of the T cell lineage are engineered with a T cell receptor (TCR) or a chimeric antigen receptor (CAR) to confer specificity to a tumor-associated antigen.

The disclosure also provides a use of cells of the T cell lineage, optionally progenitor T cells or mature T cells, generated by the methods described herein for treating a subject with cancer, wherein the cells of the T cell lineage are engineered with a T cell receptor (TCR) or a chimeric antigen receptor (CAR) to confer specificity to a tumor-associated antigen. Optionally, the iPSCs can be derived from T cells with a known or unknown TCR specificity (for example, T cells bearing TCRs with specificity against cancer), and these T-iPSCs can then be used to generate T cells by the methods described herein.

Sequences

SEQ ID NO: 1-amino acid sequence for wildtype (WT) human DLL4 starting at
residue 27:
SGVFQLQLQEFINERGVLASGRPCEPGCRTFFRVCLKHFQAVVSPGPCTFGTV
STPVLGTNSFAVRDDSSGGGRNPLQLPFNFTWPGTFSLIIEAWHAPGDDLRPEALPPD
ALISKIAIQGSLAVGQNWLLDEQTSTLTRLRYSYRVICSDNYYGDNCSRLCKKRNDHFG
HYVCQPDGNLSCLPGWTGEYCQQPICLSGCHEQNGYCSKPAECLCRPGWQGRLCNE
CIPHNGCRHGTCSTPWQCTCDEGWGGLFCDQDLNYCTHHSPCKNGATCSNSGQRS
YTCTCRPGYTGVDCELELSECDSNPCRNGGSCKDQEDGYHCLCPPGYYGLHCEHSTL
SCADSPCFNGGSCRERNQGANYACECPPNFTGSNCE.
SEQ ID NO: 2-amino acid sequence for E12 human DLL4 starting at residue 27
SSVFQLQLQEFINERGVLASGRPCEPGCRTFFRVCLKHFQAVVSPGPCTFGTV
STPVLGTNSFAVRDDSSGGGRNPLQLPLNFTWPGTFSLIIEAWHAPGDDLRPEALPPD
ALISKFAIQGSLAVGQNWLLDEQTSTLTRLRYSYRVICSDNYYGDNCSRLCKKRNDYFG
HYVCQPDGNPSCLPGWTGEYCQQPICLSGCHEQNGYCSKPAECLCRPGWQGRLCN
ECIPHNGCRHGTCSTPWQCTCDEGWGGLFCDQDLNYCTHHSPCKNGATCSNSGQR
SYTCTCRPGYTGVDCELELSECDSNPCRNGGSCKDQEDGYHCLCPPGYYGLHCEHS
TLSCADSPCFNGGSCRERNQGANYACECPPNFTGSNCE.
SEQ ID NO: 3-amino acid sequence for DLL4.v3 812 (N-EGF5) N3a.8 E12
human DLL4 starting at residue 27
SSVFQLQLQEFINERGVLASGRPCEPGCRTFFRVCLKHFQAVVSPGPCTFGTV
STPVLGTNSFAVRDDSSGGGRNPLQLPLNFTWPGTFSLIIEAWHAPGDDLRPEALPPD
ALISKFAIQGSLAVGQNWLLDEQTSTLTRLRYSYRVICSDNYYGDNCSRLCKKRNDYFG
HYVCQPDGNPSCLPGWTGEYCQQPICLSGCHEQNGYCSKPAECLCRPGWQGRLCN
ECIPYPGCRHGTCSTPWQCLCDEGWGGLYCDQDLNYCTHHSPCKNGATCHNSGPRS
YTCTCRPGYTGVDCELELSECDSNPCRNGGSCKDQEDGYHCLCPPGYYGLHCEHSTL
SCADSPCFNGGSCRERNQGANYACECPPNFTGSNCE .
SEQ ID NO: 4-amino acid sequence for DLL4.v3 (N-EGF5) N3a.5 E12 human
DLL4 starting at residue 27
SSVFQLQLQEFINERGVLASGRPCEPGCRTFFRVCLKHFQAVVSPGPCTFGTV
STPVLGTNSFAVRDDSSGGGRNPLQLPLNFTWPGTFSLIIEAWHAPGDDLRPEALPPD
ALISKFAIQGSLAVGQNWLLDEQTSTLTRLRYSYRVICSDNYYGDNCSRLCKKRNDYFG
HYVCQPDGNPSCLPGWTGEYCQQPICLSGCHEQNGYCSKPAECLCRPGWQGRLCN
ECIPHPGCRHGTCSTPWQCLCDEGWGGLYCDQDLNYCTHHSPCKNGATCRNSGPRS
YTCTCRPGYTGVDCELELSECDSNPCRNGGSCKDQEDGYHCLCPPGYYGLHCEHSTL
SCADSPCFNGGSCRERNQGANYACECPPNFTGSNCE.
SEQ ID NO: 5:-amino acid sequence for DLL4 site 2(N-EGF5) starting at
residue 27
SGVFQLQLQEFINERGVLASGRPCEPGCRTFFRVCLKHFQAVVSPGPCTFGTV
STPVLGTNSAVRDDSSGGGRNPLQLPFNFTWPGTFSLIIEAWHAPGDDLRPEALPPDA
LISKIAIQGSLAVGQNWLLDEQTSTLTRLRYSYRVICSDNYYGDNCSRLCKKRNDHFGH
YVCQPDGNLSCLPGWTGEYCQQPICLSGCHEQNGYCSKPAECLCRPGWQGRLCNEC
IPHPGCRHGTCSTPWQCLCDEGWGGLYCDQDLNYCTHHSPCKNGATCRNSGPRSYT
CTCRPGYTGVDCELELSECDSNPCRNGGSCKDQEDGYHCLCPPGYYGLHCEHSTLS
CADSPCFNGGSCRERNQGANYACECPPNFTGSNCE.
SEQ ID NO: 6-amino acid sequence for DLL4 site2 812(N-EGF5) starting at
residue 27
SGVFQLQLQEFINERGVLASGRPCEPGCRTFFRVCLKHFQAVVSPGPCTFGTV
STPVLGTNSFAVRDDSSGGGRNPLQLPFNFTWPGTFSLIIEAWHAPGDDLRPEALPPD
ALISKIAIQGSLAVGQNWLLDEQTSTLTRLRYSYRVICSDNYYGDNCSRLCKKRNDHFG
HYVCQPDGNLSCLPGWTGEYCQQPICLSGCHEQNGYCSKPAECLCRPGWQGRLCNE
CIPYPGCRHGTCSTPWQCLCDEGWGGLYCDQDLNYCTHHSPCKNGATCHNSGPRSY
TCTCRPGYTGVDCELELSECDSNPCRNGGSCKDQEDGYHCLCPPGYYGLHCEHSTLS
CADSPCFNGGSCRERNQGANYACECPPNFTGSNCE.
SEQ ID NO: 7-amino acid sequence for wildtype (WT) human DLL4 starting at
residue 27
SGVFQLQLQEFINERGVLASGRPCEPGCRTFFRVCLKHFQAVVSPGPCTFGTV
STPVLGTNSFAVRDDSSGGGRNPLQLPFNFTWPGTFSLIIEAWHAPGDDLRPEALPPD
ALISKIAIQGSLAVGQNWLLDEQTSTLTRLRYSYRVICSDNYYGDNCSRLCKKRNDHFG
HYVCQPDGNLSCLPGWTGEYCQQPICLSGCHEQNGYCSKPAECLCRPGWQGRLCNE
CIPHNGCRHGTCSTPWQCTCDEGWGGLFCDQDLNYCTHHSPCKNGATCSNSGQRS
YTCTCRPGYTGVDCELELSECDSNPCRNGGSCKDQEDGYHCLCPPGYYGLHCEHSTL
SCADSPCFNGGSCRERNQGANYACECPPNFTGSNCE.
SEQ ID NO: 8-amino acid sequence for E12 human DLL4 through EGF5 of
deltamax starting at residue 27
SSVFQLQLQEFINERGVLASGRPCEPGCRTFFRVCLKHFQAVVSPGPCTFGTV
STPVLGTNSFAVRDDSSGGGRNPLQLPLNFTWPGTFSLIIEAWHAPGDDLRPEALPPD
ALISKFAIQGSLAVGQNWLLDEQTSTLTRLRYSYRVICSDNYYGDNCSRLCKKRNDYFG
HYVCQPDGNPSCLPGWTGEYCQQPICLSGCHEQNGYCSKPAECLCRPGWQGRLCN
ECIPHNGCRHGTCSTPWQCTCDEGWGGLFCDQDLNYCTHHSPCKNGATCSNSGQR
SYTCTCRPGYTGVDCELELSECDSNPCRNGGSCKDQEDGYHCLCPPGYYGLHCEHS
TLSCADSPCFNGGSCRERNQGANYACECPPNFTGSNCE.
SEQ ID NO: 9-amino acid sequence for DLL4. v3 812 (N-EGF5) N3a.8 E12
human DLL4 through EGF5 of deltamax starting at residue 27
SSVFQLQLQEFINERGVLASGRPCEPGCRTFFRVCLKHFQAVVSPGPCTFGTV
STPVLGTNSFAVRDDSSGGGRNPLQLPLNFTWPGTFSLIIEAWHAPGDDLRPEALPPD
ALISKFAIQGSLAVGQNWLLDEQTSTLTRLRYSYRVICSDNYYGDNCSRLCKKRNDYFG
HYVCQPDGNPSCLPGWTGEYCQQPICLSGCHEQNGYCSKPAECLCRPGWQGRLCN
ECIPYPGCRHGTCSTPWQCLCDEGWGGLYCDQDLNYCTHHSPCKNGATCHNSGPRS
YTCTCRPGYTGVDCELELSECDSNPCRNGGSCKDQEDGYHCLCPPGYYGLHCEHSTL
SCADSPCFNGGSCRERNQGANYACECPPNFTGSNCE.
SEQ ID NO: 10-amino acid sequence for DLL4.v3 (N-EGF5) N3a.5 E12 human
DLL4 through EGF5 of deltamax starting at residue 27
SSVFQLQLQEFINERGVLASGRPCEPGCRTFFRVCLKHFQAVVSPGPCTFGTV
STPVLGTNSFAVRDDSSGGGRNPLQLPLNFTWPGTFSLIIEAWHAPGDDLRPEALPPD
ALISKFAIQGSLAVGQNWLLDEQTSTLTRLRYSYRVICSDNYYGDNCSRLCKKRNDYFG
HYVCQPDGNPSCLPGWTGEYCQQPICLSGCHEQNGYCSKPAECLCRPGWQGRLCN
ECIPHPGCRHGTCSTPWQCLCDEGWGGLYCDQDLNYCTHHSPCKNGATCRNSGPRS
YTCTCRPGYTGVDCELELSECDSNPCRNGGSCKDQEDGYHCLCPPGYYGLHCEHSTL
SCADSPCFNGGSCRERNQGANYACECPPNFTGSNCE.
SEQ ID NO: 11-amino acid sequence for DLL4 site 2(N-EGF5) through EGF5
of deltamax starting at residue 27
SGVFQLQLQEFINERGVLASGRPCEPGCRTFFRVCLKHFQAVVSPGPCTFGTV
STPVLGTNSAVRDDSSGGGRNPLQLPFNFTWPGTFSLIIEAWHAPGDDLRPEALPPDA
LISKIAIQGSLAVGQNWLLDEQTSTLTRLRYSYRVICSDNYYGDNCSRLCKKRNDHFGH
YVCQPDGNLSCLPGWTGEYCQQPICLSGCHEQNGYCSKPAECLCRPGWQGRLCNEC
IPHPGCRHGTCSTPWQCLCDEGWGGLYCDQDLNYCTHHSPCKNGATCRNSGPRSYT
CTCRPGYTGVDCELELSECDSNPCRNGGSCKDQEDGYHCLCPPGYYGLHCEHSTLS
CADSPCFNGGSCRERNQGANYACECPPNFTGSNCE.
SEQ ID NO: 12-amino acid sequence for DLL4 site2 812(N-EGF5) through
EGF5 of deltamax starting at residue 27
SGVFQLQLQEFINERGVLASGRPCEPGCRTFFRVCLKHFQAVVSPGPCTFGTV
STPVLGTNSFAVRDDSSGGGRNPLQLPFNFTWPGTFSLIIEAWHAPGDDLRPEALPPD
ALISKIAIQGSLAVGQNWLLDEQTSTLTRLRYSYRVICSDNYYGDNCSRLCKKRNDHFG
HYVCQPDGNLSCLPGWTGEYCQQPICLSGCHEQNGYCSKPAECLCRPGWQGRLCNE
CIPYPGCRHGTCSTPWQCLCDEGWGGLYCDQDLNYCTHHSPCKNGATCHNSGPRSY
TCTCRPGYTGVDCELELSECDSNPCRNGGSCKDQEDGYHCLCPPGYYGLHCEHSTLS
CADSPCFNGGSCRERNQGANYACECPPNFTGSNCE.

A number of embodiments of the invention have been described. Nevertheless, it will be understood that various modifications may be made without departing from the spirit and scope of the invention. Accordingly, other embodiments are within the scope of the following claims.

### EXAMPLES

### Example 1: An Affinity-Matured DLL4 Ligand for Broad-Spectrum Activation and Inhibition of Notch Signaling

### INTRODUCTION

The Notch pathway is a conserved signaling system that regulates metazoan cell fate decisions. In mammals, the core Notch signaling network consists of four Notch receptor paralogs (Notch1-4) and the activating ligands Delta-like 1 (DLL1), Delta-like 4 (DLL4), Jagged1 (JAG1), and Jagged2 (JAG2). Notch signaling occurs when cells expressing Notch proteins (signal receivers) interact with adjacent cells expressing DLL or JAG ligands (signal senders). Following ligand engagement, endocytosis of the DLL or JAG protein into the sender cell exerts a “pulling” force that destabilizes the negative regulatory region (NRR) of the Notch extracellular domain (ECD). This mechanical tension exposes a proteolytic cleavage site (S2) that is processed by the ADAM10 metalloprotease. ADAM10 cleavage results in the shedding of the Notch ECD, which in turn sensitizes the Notch transmembrane domain to cleavage by the intramembrane protease gamma-secretase. This second proteolytic event releases the Notch intracellular domain (NICD) from the plasma membrane and it translocates to the nucleus to serve as a transcriptional cofactor.

Notch ECDs are comprised of 29 to 36 epidermal growth factor (EGF) domains and the juxtamembrane negative regulatory region (NRR). Ligand ECDs also have a modular structure and contain an N-terminal C2-like (MNNL) domain, a Delta/Serrate/Lag3 (DSL) domain, 6 to 16 EGF domains, and a cysteine rich domain that is only present in JAG proteins. Structural, biochemical, and cell-based studies have determined that the C2, DSL, and EGF1-3 region of DLL or JAG engage five centrally positioned Notch EGF domains (EGF8-12 in Notch1) to initiate signaling. Notch:ligand interactions are low affinity, and co-crystallization of rat DLL4:Notch1 and JAG1:Notch1 complexes required the incorporation of affinity-enhancing mutations into the ligands. Despite this weak binding, Notch signaling occurs productively in vivo, because receptor-ligand interactions may be strengthened by catch bond formation or multivalent interactions.

Although the Notch transcriptional machinery is conserved, preferential interactions may occur between receptor and ligand subtypes. For example, DLL4 binds to Notch1 with higher affinity than DLL1, and DLL1 activates Notch1 and Notch2 equivalently¹⁵. Signaling through specific Notch-ligand pairs is also associated with distinct functional outcomes. During angiogenesis, Notch1: DLL4 interactions inhibit tip sprouting and Notch1:JAG1 interactions promote vessel growth¹⁸, and Jagged2:Notch3 interactions stimulate the differentiation of γ/δ T cells. Comparative studies have begun to identify differences in the structures, binding affinities, and signaling dynamics of selected ligands that may contribute to their unique functional properties. However, the precise molecular mechanisms controlling receptor- and ligand-specific signaling remain unclear.

Depending on cellular context, Notch signaling may induce the differentiation or proliferation of stem cells, and the broad influence of Notch on stem cell behavior has made the pathway an attractive target for regenerative therapeutics. Moreover, Notch signaling has context-dependent tumor suppressor or oncogenic functions in human cancers. The pleiotropic effects of Notch signaling suggest that both agonists and antagonists of Notch signaling will be valuable for biomedical applications. Several antagonists are currently in clinical trials for the treatment of cancer, including gamma-secretase inhibitors and monoclonal antibodies targeting individual Notch ECDs. On the other hand, the development of Notch agonists has been challenging because soluble drugs cannot exert the mechanical force necessary for receptor activation. Thus far, a single agonist antibody has been described for Notch3, which may be uniquely susceptible to antibody-mediated activation due to the inherent instability of the Notch3 NRR.

Recombinant DLL or JAG ECDs are attractive “one-size-fits-all” candidates for activating or inhibiting Notch signaling. In their soluble form, ligand ECDs act as untethered decoys that bind to Notch receptors and inhibit ligand-mediated activation. The on-target specificity of ligand ECDs may also be advantageous given that gamma-secretase cleaves a myriad of cellular substrates that are unrelated to the Notch pathway. Alternatively, surface-tethered DLL or JAG ligands stimulate Notch signaling, presumably because immobilization provides sufficient resistance force for receptor activation. Despite their potential to function as universal Notch modulators, the intrinsically low affinities of DLL and JAG ligands limits their practical utility. Additionally, the preferential binding observed between certain receptor:ligand pairs may restrict the activity of a given ligand to a subset of Notch paralogs.

To overcome the biochemical limitations of natural Notch ligands, a broadly-reactive human DLL4 variant was engineered, named Delta^{MAX}, that binds with greatly enhanced affinity to human and murine Notch receptors. Delta^{MAX} is a more potent Notch activator than WT DLL4 in several commonly used ligand-presentation formats. Furthermore, soluble Delta^{MAX} functioned as a potent, “pan-Notch” inhibitor by competing for the Notch ligand-binding site. These collective insights demonstrate that affinity-matured Notch ligands are highly-effective, multifunctional tools for modulating Notch signaling.

### Results

Structure-guided engineering of high-affinity DLL4 variants.

A structure-guided engineering strategy was employed to evolve broadly-reactive, high-affinity DLL4 variants. Structures of rat DLL4:Notch1 and JAG1:Notch1 complexes revealed that the ligand C2 and DSL domains engage the Notch1 EGF12 and EGF11 domains, respectively. In these structures, the C2:EGF12 interface was named Site 1 and the DSL:EGF11 interface was named Site 2. However, an additional “Site 3” binding interface was also visualized in JAG1:Notch1 and forms between JAG1 EGF1-3 and Notch1 EGF8-10. Site 3 contributes substantially to the affinity between Notch1 and JAG1 in solution, but has a minimal effect on the binding between DLL4 and Notch1. Therefore, a yeast display mutant library was designed to select for DLL4 mutations that recapitulate the Site 3 interaction observed in Notch1:JAG1. The library varied nine DLL4 residues (H256, N257, T271, L279, F280, T289, S301, N302, Q305) analogous to Site 3 interface residues in JAG1. A “light mutagenesis” approach was employed in which each residue was allowed to encode for the WT DLL4 residue, the equivalent JAG1 residue, or biochemically similar residues (FIG. 1A, 1B).

The DLL4 library was stained with Notch1 or Notch3 ECD constructs containing the ligand-binding domains (Notch1 EGF8-12, Notch3 EGF5-12) and selections were performed to isolate high-affinity binders. The Notch3-selected yeast had the greatest enrichment of Notch-binders, and sequencing revealed twenty-one different mutations (FIG. 1C, 7). A clone, DLL4.v2, containing five mutations (N257P, T271L, F280Y, S301R, and Q305P) was overrepresented in the samples. Analysis of the DLL4.v2 sequence revealed that F280Y and S301R are novel mutations that are not present in other Notch ligands (FIG. 8), and that N257P, T271L, and Q305P had converted to the JAG1 residues. The other four positions (DLL4 residues H256, L279, T289, and N302) reverted to the wild-type sequence (FIG. 1C). Notably, N257P was previously introduced into DLL4 to recreate the “DOS motif” found in JAG1, and this substitution increased receptor binding and signaling.

The DLL4.v2 mutations were analyzed in the context of Site 3 of the Notch1:JAG1 complex structure to gain insight into their mechanisms of affinity-enhancement (FIGS. 1D, 9). It was predicted that the N257P, T271L, and Q305P mutations enhance binding by improving hydrophobic packing at the binding interface (FIG. 9A, 9B, 9C). The F280Y mutation may stabilize the fold of the DLL4 protein by replacing the exposed hydrophobic Phe²⁸⁰ phenyl group with a more hydrophilic tyrosyl group (FIG. 9D). Lastly, it was predict that S301R enhances binding by introducing contacts between the guanidinium group of DLL4 Arg³⁰¹ and the main-chain carbonyl of the Notch1 Cys³²¹ or the aliphatic side chain of Val³²² (FIG. 9E).

### Generation of a High-Affinity DLL4 Consensus Variant.

Multiple sets of affinity-enhancing mutations were combined to engineer a DLL4 protein with maximal receptor-binding affinity. High-affinity DLL4.v2 variant was used the as a starting point for the construct design. We then engrafted five affinity-enhancing mutations (G28S, F107L, N118I, H194Y, and L206P) from previously reported rat DLL4 variant (E12) onto the human DLL4.v2 scaffold (FIG. 2A). The resulting consensus variant, Delta^{MAX}, contains 10 total mutations: G28S, F107L, N118I, H194Y, L206P, N257P, T271L, F280Y, S301R, and Q305P (FIG. 2A).

DLL4 Variants Bind to Human and Mouse Notch Receptors with High-Affinity.

Surface plasmon resonance (SPR) was used to determine the binding affinity between Delta^{MAX} and the ligand-binding regions of Notch1-4. As a basis for comparison, a WT DLL4 construct, a DLL4.v2 construct, and a “DLL4.v1” construct containing the five E12 mutations (G28S, F107L, N118I, H194Y, and L206P) were also generated (FIG. 2A, 10). Analysis of the SPR data revealed that DLL4.v1, DLL4.v2, and Delta^{MAX} bound to all four human Notch receptors with enhanced affinity compared to WT DLL4 (FIG. 2B). Delta^{MAX} bound to Notch1 with a dissociation constant (K_{D}) of 24 nM, Notch2 with a K_{D} of 54 nM, Notch3 with a K_{D} of 49 nM, and Notch4 with a K_{D} of 24 nM. Compared to WT DLL4, the nanomolar affinity interactions between Delta^{MAX} and Notch1-4 represent enhancements of 1,029-fold, 670-fold, 591-fold, and 532-fold, respectively. The rapid dissociation of WT DLL4 precluded kinetic fitting of the binding data. However, fitting the SPR sensograms for DLL4.v1, DLL4.v2, and Delta^{MAX} indicated that the increasing affinities are mostly due to progressive decreases in off-rate (FIG. 2C).

The binding affinity was measured between each DLL4 variant and the EGF6-13 region of murine Notch1 (mNotch1 EGF6-13) to test for cross-species reactivity (FIGS. 2B, 10, 11A). Each ligand bound to murine Notch1 and human Notch1 with comparable affinities, and Delta^{MAX} bound to mNotch1 EGF6-13 with a K_{D} of 25 nM. Lastly, the binding of Delta^{MAX} to full-length, cell surface-expressed Notch1 was assessed. The mean fluorescence intensity (MFI) of Delta^{MAX} binding to Notch1-overexpressing cells increased by 4.7-fold compared to WT DLL4 (FIG. 11C, 11D).

### Delta^{MAX} has Improved Expression and Thermostability.

Whether the affinity-enhancing mutations impacted the stability and expression of Delta^{MAX} was tested. A thermal denaturation experiment was performed using differential scanning fluorimetry (DSF) to compare the melting temperatures of WT DLL4 and Delta^{MAX}. It was determined that the melting temperature (Tₘ) of WT DLL4 was 47° C. and that the Tₘ of Delta^{MAX} was increased by 11° C. to 58° C. (FIG. 2D). Furthermore, expression yield of the Delta^{MAX} protein was increased by 2-fold compared to WT DLL4 (FIG. 10G, 10H, 10I). Taken together, our biochemical experiments indicate that Delta^{MAX} exhibits enhanced affinity, stability and expression while maintaining a broad reactivity profile against human and murine Notch receptors.

Delta^{MAX} Activates Notch More Potently than WT DLL4.

The signaling potency of Delta^{MAX} was first characterized using fluorescent Notch1 reporter cells (FIG. 11D, 12A, 13A). As the goal was to develop an accessible tool for modulating Notch activity, this assay was performed by non-specifically adsorbing equivalent amounts of DLL4 proteins to tissue culture plates (FIG. 11B). This facile method can be performed with unmodified proteins and is an established approach for stimulating Notch in vitro. Notch1 reporter cells were cultured on the ligand-coated surfaces, and after 24 hours, fluorescence was monitored by flow cytometry. The EC50 values for Notch1 activation by WT DLL4 and Delta^{MAX} were 2.3 ng/mm² and 0.009 ng/mm², respectively, which corresponds to a 250-fold improvement in Notch activation efficiency (FIG. 3A). Moreover, the Eₘₐₓ induced by Delta^{MAX} was 20% greater than that induced by WT DLL4.

Next tested was whether C-terminal anchoring of DLL4 proteins improves signaling output. This strategy mimics the orientation the proteins adopt on the cell surface and was achieved by biotinylating the ligand C-termini prior to attachment to streptavidin-coated plates. For both WT DLL4 and Delta^{MAX}, oriented coupling induced a higher maximum level of Notch1 activation than non-specific adsorption (FIG. 3B). In this format, Delta^{MAX} was also more potent than WT DLL4 and activated Notch1 with an EC50 of 1.7 nM compared to 16 nM for WT DLL4. A time-course experiment was also performed to evaluate the signaling kinetics of Delta^{MAX}. It was determined that Delta^{MAX} activates Notch1 more rapidly than WT DLL4, and that this effect is most pronounced in the first 4 to 8 hours following ligand stimulation (FIG. 3C).

### Delta^{MAX} Strongly Activates Multiple Human Notch Receptors.

The ability of Delta^{MAX} to activate Notch1, Notch2, and Notch3 was also tested using an established luciferase reporter assay (FIG. 11D, 12B). Notch4 was excluded from the study since it is reportedly unresponsive to ligand-mediated activation. Immobilized Delta^{MAX} activated Notch1-3 more potently than WT DLL4 across all concentrations tested. At the highest concentration of DLL4 or Delta^{MAX} (50 nM), Notch1, Notch2, and Notch3, signaling was increased by 2-fold, 2.5-fold and 3-fold, respectively (FIG. 3D).

To determine whether Delta^{MAX} stimulates increased activation in cells expressing endogenous levels of Notch1, we compared the signaling of Delta^{MAX} and WT DLL4 in U2OS (osteosarcoma) and MCF-7 (breast cancer) cells. U2OS and MCF-7 cells were each stimulated for 24 hours using C-terminally anchored WT DLL4 and Delta^{MAX} and Notch1 activation was detected by Western blotting for the cleaved Notch1 ICD (FIG. 3E, FIG. 11D). Delta^{MAX} activated Notch signaling much more potently than WT DLL4 in both cell lines, indicating that Delta^{MAX} is a more effective agonist regardless of Notch expression level.

To assess the activity of Delta^{MAX} when expressed in cells, a co-culture Notch signaling assay was performed. HEK293T cell lines stably expressing either full-length WT DLL4 or Delta^{MAX} (FIG. 11B) were generated and co-cultured with Notch1, Notch2, or Notch3 reporter cells (FIG. 12B) at a 1:1 ratio. The Eₘₐₓ of Delta^{MAX} signaling through Notch1 and Notch3 was ˜2-fold higher than WT DLL4. The luciferase signal for Notch2 was low for both ligands, although only Delta^{MAX} induced a significant level of Notch2 activation compared to unstimulated cells (P=0.037) (FIG. 3F).

### Comparison of Delta^{MAX} Signaling on Plates, Beads, and Cells.

The levels of Notch signaling induced by different ligand-presentation formats have not previously been directly compared in a single study. Here, Delta^{MAX} was evaluated in five different formats to determine an optimal strategy for maximizing Notch activation. Notch1 reporter cells were cultured with yeast-displayed Delta^{MAX}, 293T cells-expressing Delta^{MAX}, Delta^{MAX}-coated microbeads (1-10 μm), plates coated with Delta^{MAX} (non-specific coupling), and streptavidin plates coated with C-terminally biotinylated Delta^{MAX} (oriented coupling) (FIGS. 3G, 14). Yeast-displayed Delta^{MAX} did not induce a significant change in Notch1 activation. However, the remaining formats induced 15- to 50-fold increases in Notch1 activation relative to controls. The co-culture method induced the lowest level of Notch1 activation (15-fold), followed by Delta^{MAX}-coated beads (21-fold), non-specific adsorption (23-fold), and oriented coupling (50-fold) (FIG. 3G). Taken together, our data reveal that recombinant protein-based formats (plate- or bead-bound ligands) induced higher signaling levels than cell-based methods. Furthermore, the increased Eₘₐₓ of C-terminally anchored Delta^{MAX} suggests that the greater accessibility of the ligand-binding C2-EGF3 domains is ideal for maximizing Notch stimulation.

The size of DLL4-coated microbeads was recently reported to be a key determinant of signaling activity, with 25 μm beads being associated with maximal levels of Notch activation in T cells. Therefore, Notch1 activation was compared by 25 μm beads coated with DLL4 and Delta^{MAX}. Delta^{MAX} microbeads induced 4.5-fold higher reporter activity than DLL4 microbeads (FIG. 14E), which is consistent with the increased signaling of Delta^{MAX} across presentation formats.

### Soluble Delta^{MAX} is a Potent Pan-Notch Inhibitor.

In the absence of membrane anchor, DLL4 ECDs are predicted to block ligand-mediated Notch activation by functioning as soluble decoys. To test whether DLL4 variants function as effective inhibitors, CHO-K1 N1-Gal4 reporter cells were cultured on WT DLL4-coated plates in the presence of soluble competitors. Increasing concentrations of WT DLL4, DLL4.v1, DLL4.v2, and Delta^{MAX} ECDs were added to the cells and then reporter signal monitored after 24 hours. Among the four proteins, only soluble Delta^{MAX} efficiently blocked Notch1 activation and inhibited signaling with an IC50 of 0.6 nM (FIG. 4A). By contrast, administration of soluble WT DLL4 only reduced reporter activity by ˜25% at the highest concentration tested (300 nM).

Most small molecule Notch inhibitors are non-specific and target multifunctional ADAM10/17 or gamma-secretase proteases required for Notch activation. Here, the inhibition potency of the Notch-selective Delta^{MAX} protein were compared to the gamma-secretase inhibitor DAPT and the metalloprotease/ADAM10 inhibitor BB-94 (FIG. 4B). It was determined that Delta^{MAX} inhibited Notch1 activation with an IC50 of 1 nM, which was 40-fold more potent than DAPT (IC50 of 40 nM) and 1,000-fold more potent than BB-94 (IC50 of 1,000 nM). The inhibition of Delta^{MAX} and DAPT was also compared in a co-culture assay. Both DAPT and Delta^{MAX} inhibited cellular DLL4 less potently than plate-bound DLL4, and the IC50 of DAPT and Delta^{MAX} were 100 and 400 nM, respectively (FIG. 15). Thus, Delta^{MAX} functions as a potent and selective inhibitor of ligand-mediated Notch signaling, and the potency of inhibition depends on the format in which the wild-type ligand is presented.

The ability of Delta^{MAX} to inhibit signaling by ligands other than DLL4 was also tested. Stable HEK293T cell lines expressing DLL1, DLL4, JAG1, and JAG2 (FIG. 16) were generated and co-cultured with Notch3 reporter cells that were pre-mixed with a constant (3 μM) concentration of soluble WT DLL4 or Delta^{MAX} (FIG. 4C). It was determined that Delta^{MAX} strongly inhibited signaling by DLL1, DLL4, JAG1, and JAG2, with a reduction in reporter signal ranging from 70-85%. On the other hand, WT DLL4 only reduced reporter signal by 10-40%.

Lastly, whether Delta^{MAX} could inhibit signaling through different Notch paralogs was assessed. Notch1, Notch2 or Notch3 reporter cells were incubated with various concentrations of soluble WT DLL4 or Delta^{MAX} and cultured on plates coated with DLL4-coated plates. Addition of the Delta^{MAX} competitor led to dose-dependent decreases in signaling for Notch1, Notch2, and Notch3 (FIG. 4D).

Delta^{MAX} Increases Proliferation and Expression of Effector Markers in CD8⁺ T Cells.

Notch signaling contributes to several aspects of immunobiology and influences the proliferation, differentiation, and antitumor function of CD8⁺ T cells. Therefore, an assay was developed to test the effect of Delta^{MAX} on activated T cells (FIG. 5A). As stromal cells are an established platform for Notch stimulation in T cells, artificial antigen presenting K562 cells (aAPCs) expressing the Fc receptor CD32 (renamed K32 cells), and similar surface levels of WT DLL4 or Delta^{MAX} (FIG. 5B) were generated. these ligand-expressing cells were then co-cultured with primary T cells in the presence of increasing concentrations of the T cell-activating antibody OKT3. CD32 expression enabled OKT3 Fc binding to K32 cells and subsequent CD3-TCR activation on T cells. Human CD8⁺ T cells were negatively isolated from peripheral blood and co-cultured with K32 cells pre-loaded with increasing concentrations of OKT3 to analyze T cell proliferation and IFNγ secretion (FIGS. 5C, 5D). Delta^{MAX} treatment enhanced the percentage of proliferating CD8⁺ T cells between 27% and 15% along the OKT3 concentrations assayed (P=0.032), while WT DLL4 did not show a significant improvement over the stimulation with K32 cells lacking DLL4 ligands (mock) (FIG. 5C). Additionally, Delta^{MAX} increased IFNγ secretion from CD8⁺ T cells 10-15% compared with WT DLL4 treatment (P=0.047) (FIG. 5D).

Next, RT-PCR was used to quantify the mRNA levels of Notch and T cell effector related markers over four days of CD8⁺ T cell stimulation with K32 loaded with 0.0125 μg/mL OKT3. It was determined that Delta^{MAX}-stimulated T cells had increased levels of effector mediators IFNγ and Granzyme B, as well as elevated expression of Notch target Hes-4 compared to counterparts conditioned with WT DLL4 or mock (FIG. 5E). Finally, a Western blot analysis was performed to compare Notch1 and Notch2 activation in T cells that were stimulated with WT DLL4 or Delta^{MAX}. Using an antibody to the cleaved Notch1 or Notch2 ICD, it was determined that activation of both Notch1 and Notch2 was increased for Delta^{MAX}-treated cells relative to WT DLL4. The difference in Notch2 activation was more prominent than that of Notch1, which was attributed to the higher expression of Notch2 in stimulated T cells as detected by flow cytometry (FIGS. 5F, 11E). Thus, our results show the promotion of CD8⁺ T cell function induced by aAPCs expressing Delta^{MAX}.

Soluble Delta^{MAX} Promotes the Differentiation of Neuronal Progenitors into Neurons.

The next goal was to determine whether soluble Delta^{MAX} inhibits Notch in a physiological system. Notch signaling helps maintain undifferentiated neural progenitors in a proliferative state, and Notch inhibition has been implicated in differentiating neural progenitors into neurons, both in vivo and in vitro (FIG. 6A). Soluble Delta^{MAX} enhanced the differentiation of human pluripotent stem cells (hPSCs) into cerebral cortex neurons. First, hPSCs were differentiated into neural progenitors for 7 days, using a modification of a previous method. Second, hPSC-derived neural progenitors were further differentiated into cerebral cortex neurons for 7 additional days, in the presence of soluble Delta^{MAX}, soluble WT DLL4, DAPT, or the absence of any Notch modulator. Without Notch inhibition, the neural progenitor marker SOX2 was expressed, and neuronal markers were not upregulated (FIG. 6B). Critically, soluble Delta^{MAX} enhanced differentiation into cerebral cortex neurons in a concentration-dependent way: it repressed SOX2 and sharply increased pan-neuronal markers (MAPT, SNAP25, DCX; FIG. 6C, 6D, 6E), glutamatergic markers (VGLUT2, FIG. 6F) and cerebral cortex neuronal markers (TBR1, FIG. 6G). 1 μM of soluble Delta^{MAX} was comparable to, if not superior to, 10 μM of DAPT in eliciting neuronal differentiation. Importantly, soluble WT DLL4 failed to upregulate neuronal markers or to repress neural progenitor markers, thus demonstrating the superiority of soluble Delta^{MAX}. Taken together, soluble Delta^{MAX} is a potent Notch pathway antagonist that drives differentiation of hPSC-derived neural progenitors into neurons.

### Discussion

Recombinant DLL or JAG ECDs bind weakly to Notch receptors and are inefficient modulators of Notch signaling. The synthetic Delta^{MAX} ligand was engineered to be a versatile tool for activating or inhibiting mammalian Notch receptors. Compared to WT DLL4, immobilized or cellular Delta^{MAX} exhibits varying degrees of increased signaling potency, and soluble Delta^{MAX} functioned as a Notch-specific inhibitor when administered as a soluble decoy. Furthermore, the increased thermostability (ΔTₘ 11° C.) and expression (2-fold higher yield) of Delta^{MAX} are favorable properties for protein biologics. Delta^{MAX} was generated, in part, by engineering the Site 3 binding interface observed in the Notch1:JAG1 structure. The Site 3 region is functionally important for DLL4 signaling despite its minimal contribution to the solution binding affinity between DLL4 and Notch1 ECDs. This suggests that additional biophysical factors, such as clustering or ‘pulling forces’ associated with ligand endocytosis, may enforce Site 3 interactions in Notch ligands.

The largest difference in signaling potency between WT DLL4 and Delta^{MAX} was observed when ligands were non-specifically adsorbed to plastic. The WT DLL4 protein was a poor Notch activator in this format (EC50 2.3 ng/mm²), while Delta^{MAX} was remarkably potent (EC50 0.009 ng/mm²). The strong signaling activity can enable entire flasks or tissue culture plates to be pre-conditioned with Delta^{MAX} as a high-throughput means of activating Notch in cultured cells. Furthermore, non-specific adsorption requires low quantities of protein and can be performed with unmodified ligand ECDs, making this method highly accessible for biotechnological applications.

Even at high-concentrations, the micromolar binding affinity of the WT DLL4 ECD makes it an ineffective antagonist. By contrast, soluble Delta^{MAX} potently inhibited Notch activation by DLL1, DLL4, JAG1, and JAG2 and effectively inhibited Notch-dependent differentiation of neuronal precursors. Antibodies targeting the NRR domain have been used to block signaling by individual Notch receptors, which was associated with reduced gastrointestinal toxicity compared to gamma-secretase or Notch1/2 dual-inhibition. Alternatively, pan-Notch inhibitors, such as DAPT or BB-94, non-specifically block cleavage of numerous substrates besides Notch. Therefore, the ability of Delta^{MAX} to selectively inhibit all human Notch receptor paralogs distinguishes it among our toolkit of Notch-modulating molecules.

It was previously demonstrated that constitutive Notch activation by overexpression of NICD enhances the antitumor activity of adoptively transferred murine CD8⁺ T cells. In the present study, an “off-the-shelf” approach was developed that transiently activates Notch using Delta^{MAX}-expressing K32 cells. This circumvents the requirement for constitutive Notch activation and may be advantageous since Notch hyperactivation has been associated with oncogenic transformation in T cells. Co-culture with K32-Delta^{MAX} may also be beneficial for strategies of expansion of anti-tumor CAR-T or TILS for further adoptive transfer by triggering Notch-related metabolic and anti-tumor signals.

Various ligand presentation strategies have been developed for activating Notch in T cells, and in these systems, the dosage was important for achieving optimal outcomes. For example, low doses of DLL1 stimulate the differentiation of hematopoietic stem and progenitor cells into lymphoid and myeloid precursors, whereas high-doses promote the differentiation of only lymphoid precursors. Additionally, stromal cells that express DLL4, which binds to Notch1 with higher affinity than DLL1, more efficiently drive T cell lymphopoiesis. Given these tunable outcomes, the ultra-potent Delta^{MAX} ligand may be used to optimize Notch activation for T cell-related applications.

### Material and Methods

### Reagents, Media, and Cell Lines

Mammalian cells were cultured at 37° C., with a humidified atmosphere of 5% CO₂, washed with Dulbecco's Phosphate Saline Buffer (DPBS, Corning), and detached with Trypsin-EDTA 0.25% (Gibco) for cell sub-culturing or cell-based assays. Notch reporter cell lines CHO-K1 N1-Gal4 were provided (California Institute of Technology). HEK293T, U2OS and MCF-7 cells were cultured in high-glucose Dulbecco Eagle's Minimal Essential Medium (DMEM, Cytiva) supplemented with 10% Fetal Bovine Serum (FBS, peak serum) and 2% penicillin/streptomycin (Gibco). The MCF-7 breast cancer cell line was a gift from Dr. Eric Lau (Moffitt Cancer Center). Hygromycin 100 μg/ml was added to U2OS reporter cell cultures to maintain homogeneous populations of Notch-expressing cells. CHO-K1 N1-Gal4 cells were cultured in Minimum Essential Medium Eagle-alpha modification (α-MEM, Cytiva) supplemented with 10% Fetal Bovine Serum (FBS, peak serum), 2% penicillin/streptomycin (Gibco), 400 μg/mL of Zeocin (Alfa aesar) and 600 μg/mL of Geneticin (Gibco). K32 cells (K562 cell line expressing CD32) were a gift from Dr. Jose Conejo-Garcia (Moffitt Cancer Center), and human CD8⁺ T cells were PBMCs (collected from LifeSouth Blood Bank, Gainesville, FL). Cells used for T cell experiments were cultured at 37° C., and 5% CO₂, in RPMI-1640 (Gibco) supplemented with 2 mM L-glutamine, 10 mM HEPES, 150 U/mL streptomycin, 200 U/mL Penicillin, 20 mM β-mercaptoethanol and 10% heat-inactivated FBS (GeminiBio). All cell lines were tested negative for mycoplasma (ATCC), and stable cell lines were validated by sequencing (Genewiz) after genomic DNA extraction (Invitrogen). Antibodies used in this study were: anti-hNotch1 PE (Biolegend), anti-hNotch2 AF647 (Biolegend), anti-hNotch3 PE (Biolegend), anti-hDLL4 PE (Biolegend), anti-hDLL1 PE (Biolegend), anti-hJagged1 AF647 (Biolegend), anti-hJagged2 PE (Biolegend), and anti-HA tag AF488 (Cell Signaling Technologies), anti-hCD8a Bv785 (Biolegend), anti-hIFNγ APC (Biolegend), goat anti-mouse IgG (secondary antibody, KPL), goat anti-rabbit IgG (secondary antibody, Vector laboratories), anti-cleaved Notch1 (Val1744, Cell Signaling Technology), anti-Notch2 (D76A6, Cell Signaling Technologies), anti-β-Actin (Cell Signaling Technology and Sigma), anti-6×His Tag (Bethyl).

### Protein Expression and Purification

The minimum binding region of Delta-Like 4 (DLL4) fragments (N-EGF5) were cloned into the pAcGp67A vector containing an N-terminal gp67 signal peptide and C-terminal 8×His-tag. Non-biotinylated proteins were human wild-type DLL4 (N-EGF5, residues 27-400), DLL4.v1 (N-EGF5), DLL4.v2 (N-EGF5), and DLL4^{MAX} (N-EGF5). Notch biotinylated fragments were cloned into pAcGp67A with a N-terminal gp67 signal peptide, a C-terminal 3C protease site followed by biotin-acceptor peptide tag (BAP-tag: GLNDIFEAQKIEWHE), and 6×His-tag. In contrast, DLL4 biotinylated proteins contained BAP and His-tags, but lacked 3C site. Biotinylated proteins were human Notch1 (EGF8-12, residues 295-488), hNotch1 (EGF6-13, residues 237-549), hNotch2 (EGF6-13, residues 221-530), hNotch3 (EGF5-12, residues 197-505), hNotch4 (EGF7-12, residues 276-511), mouse Notch1 (EGF6-13, residues 221-526), WT DLL4 (N-EGF5, residues 27-400), and DLL4^{MAX} (N-EGF5, residues 27-400). All baculovirus-based constructs in this work were expressed for 48 hours by infecting HiFive Trichoplusia ni insect cell cultures (Expression Systems) at a density of 2×10⁶ cells/mL with infective baculovirus particles. Culture supernatants were harvested, and proteins purified by IMAC. Ni-NTA agarose resin (Qiagen) was washed with HEPES Buffered Saline (HBS: 20 mM HEPES pH 7.4, 150 mM sodium chloride; Buffer A) supplemented with 25 mM imidazole (plus 1 mM calcium chloride for Notch preps; Buffer A+C) and eluted with Buffer A (or Buffer A+C for Notch samples) containing 250 mM imidazole. Polishing was performed using a Superdex 200 Increase 10/300 GL column (GE) equilibrated in Buffer A for DLL4 proteins, or Superdex 75 Increase 10/300 GL column (GE) equilibrated in Buffer A+C for Notch proteins. Biotinylated proteins were site-specifically modified in vitro at their C-terminal BAP-tag, using BirA ligase (in-house prepared) and polished as described above. Protein purity and integrity were analyzed by SDS-PAGE using TGX 12% Pre-cast gels (Bio-Rad). All proteins were flash frozen in liquid nitrogen and stored at −80° C. for later use.

### Affinity Maturation of Human DLL4 by Directed Evolution

### Starting Point of Evolution and Structure-Guided Library Creation

Wild-type human Delta-like 4 (N-EGF3, residues 27-322) was cloned into a modified version of pCT302 an N-terminal fusion to an HA-epitope (YPYDVPDYA, SEQ ID NO: 13), c-Myc epitope (EQKLISEEDL, SEQ ID NO:14), and Aga2. A third binding site for DLL4:Notch interaction was designed on the basis of the binding interface found in the crystal structure of rJagged1(N-EGF3):rNotch1(EGF8-12) complex. The complex was analyzed with different computational tools (PDBePISA), Consurf server (Biosof LLC), and PyMOL (The PyMOL Molecular Graphics System, Version 2.0 Schrödinger, LLC)). Nine interface residues were selected to create a structure-guided directed evolution library. Jagged1 homolog residues were identified in DLL4 and targeted for mutagenesis. The mutant library was generated using assembly PCR with ten overlapping oligonucleotides.

The primers contained specific degenerate codons and 20 bp overhangs (Eurofins) to promote annealing in the PCR reaction. External primers were added to introduce a 40-50 bp recombination area between the mutant library region (EGF1-EGF3) and DLL4-pYAL linearized vector. The mutant library was in vivo reassembled into yeast by electroporation (BTX electroporator) with 10 μg of linearized DLL4-pYAL vector and 50 μg of the mutagenic library (ratio 1:5 vector:library). Yeast transformants were recovered in SDCAA and DLL4 surface displayed on S. cerevisiae EBY100 yeast. The theoretical library diversity was 4×10⁶, and we obtained an estimated library size of 8×10⁶ variants (2-fold theoretical diversity).

### Yeast Display Selections

Before starting each selection round, induced yeast cultures were pelleted in logarithmic phase, stained with anti-HA antibody conjugated to AF488 (Cell Signaling Technologies), and expression of DLL4 on yeast surface analyzed by flow cytometry (BD Accuri C6 Plus). Selection rounds were performed in two phases: (1) a negative selection to remove non-specific binders targeting streptavidin and/or Alexa Fluor 647, and (2) a positive selection to rescue high-affinity variants specific for Notch. Negative selection was performed by combining 100 nM of streptavidin conjugated to Alexa Fluor 647 (SA647) and 250 μL anti-Alexa Fluor 647 magnetic beads (Miltenyi) on ice for 10 min. Next, the yeast library was washed once in selection buffer (HBS: 20 mM HEPES pH 7.4, 150 mM sodium chloride, 1 mM calcium chloride, 0.1% Bovine Serum Albumin (BSA) and 10 mM maltose), and magnetic beads SA647-coated were added. The tube was wrapped in foil and incubated at 4° C. for 30 min. Following this step, the yeast was washed twice with selection buffer, resuspended, and flowed over a Magnetic-Activated cell sorting (MACS) LS separation column (Miltenyi) pre-equilibrated in selection buffer. The flowthrough was rescued, and positive selections were performed. For round zero (Naïve library), 8×10⁷ cells (10× library diversity) were used for selection against biotinylated Notch1 (EGF8-12) and Notch3 (EGF5-13), independently. Tetramers were pre-made on ice for 10 min by mixing 450 nM of biotinylated Notch1 (EGF8-12) or Notch3 (EGF5-12) with 100 nM of SA-647 (ratio 4.5:1). Next, 100 nM tetramers were added to 500 μL of negatively selected yeast in selection buffer and incubated for 2 hours at 4° C. Yeast library stained with Notch tetramers was washed twice with 500 μL of selection buffer, and further incubated with 50 μL of anti-AF647 magnetic beads for 30 min at 4° C. After incubation, the yeast was washed twice with 500 μL of selection buffer and high-affinity binders isolated by MACS columns. The isolated yeast cells were pelleted, recovered in 3 mL of SDCAA for 48 hours and induced in SGCAA for next round. For round 1, the same conditions were repeated like in round zero using Notch1 (EGF8-12) and Notch3 (EGF5-12) 100 nM tetramers for positive selections. Isolated populations were rescued in 3 mL SDCAA after MACS sorting and induced in SGCAA. For round 2, 5×10E7 yeast cells were resuspended in 500 μL of selection buffer in the presence of 2 μM monomer of Notch1 (EGF8-12) and Notch3 (EGF5-12), independently. Tubes were wrapped in foil and incubated for 2 hours at 4° C. Yeast samples binding Notch were washed twice with 500 μL of selection buffer, and incubated in 500 μL of selection buffer supplemented with 100 nM of SA-647 for 30 min at 4° C. DLL4 high-affinity binders were isolated by MACS following the same approach described for rounds zero and 1. High-affinity binders for Notch1 and Notch3 were rescued in 3 mL of SDCAA for 48 hours and induced in SGCAA. For round 3, high-affinity populations were isolated by Fluorescent-Activated Cell Sorting (FACS, Sony Sorter SH800S). Only Notch3 (EGF5-12) was used since yeast-staining tests showed better population enrichment compared with Notch1 (EGF8-12). 1×10⁸ yeast cells were resuspended into 500 μL of selection buffer with pre-made 100 nM tetramers of Notch3 (5-12) and anti-HA488 antibody (double staining). The tube was wrapped in foil and incubated for 2 hours at 4° C. Stained yeast was washed two times with 500 μL of selection buffer and analyzed by FACS. From the double-positive population (FIT-C+, APC+) showing expression of DLL4 (anti-HA488, FIT-C signal) and binding to Notch (SA-647, APC signal), a sorting gate was set at 1.8% of the maximum signal detected for APC channel and Notch binders isolated (FIG. 7B). The sorted yeast population was washed two times with 5 mL of selection buffer, pelleted, and recovered in SDCAA for 48 hours. Yeast cells were induced again in SGCAA for the last round of selection. For round 4, 5×10⁷ cells were incubated with 0.2 μM monomers of Notch3 (5-12) for 2 hours at 4° C. Yeast cells were then washed twice with selection buffer, fluorescent-labeled with SA-647 and high-affinity populations isolated by MACS following the protocol described for previous rounds.

The isolated yeast populations obtained after round 4 were subjected to colony screen and plasmid recovery by using Zymoprep kit (Zymo Research). The plasmids were transformed into E. coli and 30 individual colonies sequenced with pYAL F (5′-AAATGATAACCATCTCGC, SEQ ID NO: 15) and pYAL R (5′-GGGATTTGCTCGCATATAGTTG, SEQ ID NO:16) primers (Eurofins). Sequencing data analysis was performed using SnapGene software (Insightful Science).

### Generation of DLL4 Variants

A variant named DLL4.v1 was first generated by engrafting five affinity enhancing mutations into human DLL4 scaffold that was previously reported for rat DLL4 affinity maturation (Site 1+Site 2; G28S, F107L, I143F, H194Y, and L206P). Next, the mutations found in this work to create a third binding site on human DLL4 (Site 3; N257P, T271L, F280Y, S301R, Q305P) were combined with Site 1+Site 2 mutations into one single DLL4 scaffold, creating an ultrapotent DLL4 protein, named Delta^{MAX} (Site 1+Site2; G28S, F107L, I143F, H194Y and L206P, and Site 3; N257P, T271L, F280Y, S301R, Q305P).

### Thermal Denaturation Experiments by Differential Scanning Fluorometry (DSF)

WT DLL4 and Delta^{MAX} (N-EGF5) were diluted at 5 μM in 20 μL of HBS buffer, and then combined with 10 μM (5×) SYPRO Orange (ThermoFisher Scientific) prepared in 100% DMSO (final DMSO concentration was 0.1%). This mix was equilibrated at room temperature for 30 min. Proteins were analyzed in a 96-well microtiter plate using a StepOnePlus RT-PCR system (Applied Biosystems) applying a continuous heating gradient from 25 to 99° C. with a step of 1% of temperature/min. Data was normalized and represented as the mean of three independent technical replicates. Similar Tₘ values were obtained for the replica measurements.

### Determination of Equilibrium Dissociation Constants (K_{D}) by Surface Plasmon Resonance (SPR)

Equilibrium dissociation constants were determined by surface plasmon resonance using a Biacore T200 instrument (GE Healthcare). Approximately 100 resonance units (RU) of biotinylated Notch extracellular fragments (Notch1 (EGF6-13), Notch2 (EGF6-13), Notch3 (EGF5-12), Notch4 (7-12), and mouse Notch1 (EGF6-13)) were immobilized on individual flow cells at 5 μl/min using a streptavidin-coated sensor chip (Series S Sensor chip SA, GE Healthcare). Three-fold serial dilutions of recombinant DLL4 proteins (N-EGF5) starting at 50 μM (WT DLL4), 18 μM (DLL4.v2), and 6 μM (DLL4.v1 and Delta^{MAX}) were flowed over the chip at 25° C. in SPR buffer (20 mM HEPES pH 7.4, 150 mM NaCl, 1 mM calcium chloride, 0.1% BSA supplemented with 0.005% surfactant P20). Association (“on-rate”) and dissociation (“off-rate”) phases were performed at 10 μl/min for 120 seconds and 300 seconds, respectively. The sensor chip was regenerated after each injection with 30-second washes of 2.25M Magnesium Chloride containing 25% Ethylene Glycol at 50 μL/min. Data collection rate was performed at 10 Hz, and curves were reference-subtracted from a flow cell containing 100 RU of a negative control non-related protein (biotinylated mouse RNF43 PA domain). The maximum RU for each experiment was normalized to a value of 100% and plotted as a function of concentration using Prism 9 software (GraphPad). Steady-state binding and kinetic curves were fitted using the Biacore T200 evaluation software to a 1:1 Langmuir model to determine the K_{D} values.

### Generation of Stable Cell Lines

Ectodomains of WT DLL4 and Delta^{MAX} (N-EGF8, residues 27-518) were cloned into an intermediary vector containing WT DLL4 signal peptide, juxtamembrane, transmembrane, and intracellular domain, fused to C-terminal Myc (EQKLISEEDL) and Flag (DYKDDDDK) tags. This vector was generated after cloning of a synthetic DNA fragment (Eurofins) into pLenti-C-Myc-DDK-IRES-Puro vector (Invitrogen). For stable cell lines expressing DLL1 (residues 1-723), Jagged1 (residues 1-1218), and Jagged2 (residues 1-1238), their CDS was cloned into pLenti-C-Myc-DDK-IRES-Puro vector fused to c-Myc and Flag C-termini tags. HEK-293T mammalian cells were used for lentiviral particle production and transductions (kindly donated by Dr. Eric Lau, Moffitt Cancer Center). Briefly, transfections were carried out with packaging vectors VSV-G and d8.9 (kindly donated by Dr. Eric Lau, Moffitt Cancer Center) in the presence of Polyethyleneimine (PEI) at ratio 4:1 (DNA:PEI). Lentiviral particles were harvested after 48-72 hours and used to transduce HEK293T cells using 1 μg/mL polybrene (Millipore). Antibiotic selection was performed using Puromycin at 5 μg/mL (Gibco) for 10-15 days. Puromycin resistant cells expressing WT DLL4 and Delta^{MAX} were detached with trypsin-free dissociation buffer (Gibco), stained in DPBS+0.5% BSA with anti-hDLL4 PE antibody for 1 hour at 4° C., and then sorted by FACS (Sony sorter SH800S). Sorted cells (mean fluorescence intensity around 1×10E5-3×10E5) were washed with DPBS and recovered in DMEM+10% FBS at 37° C. and 5% CO₂ for 2 weeks. Expression of WT DLL4 and Delta^{MAX} on HEK293T surface was confirmed by flow cytometry (BD Accuri C6 plus) staining the cell lines with anti-hDLL4 PE in DPBS+0.5% BSA (FIG. 15B). DLL1, Jagged1, and Jagged2 cell lines were stained with specific antibodies (anti-hDLL1 PE, anti-hJagged1 AF647, and anti-hJagged2 PE) for 1 hour at 4° C. and analyzed by flow cytometry (BD Accuri C6 plus); however, these cell lines did not require further sorting steps (FIG. 16).

K32 cells (K562 cells expressing human CD32) were transduced with lentivirus expressing WT DLL4 or Delta^{MAX} by spin-infection using 8 μg/mL polybrene (Millipore). Seventy-two hours after transduction, cells were analyzed for DLL4 expression and further cultured in RPMI+10% FBS containing 1 μg/ml of puromycin (Gibco) at 37° C. and 5% CO₂. DLL4 expression was monitored by flow cytometry (CytoFLEX II, Beckman Coulter) using anti-hDLL4 PE (Biolegend) until the culture reached ˜100% of positive population on PE signal.

Notch Activation Assays with Ligands Non-Specifically Adsorbed to Plates

On day one, 2-fold serial dilutions of non-biotinylated DLL4 recombinant proteins (N-EGF5) were prepared in 100 μL of DPBS and used to coat 96-well plates (Coastar) for 1 hour at 37° C. WT DLL4 started at 66.56 ng/mm² and Delta^{MAX} at 0.33 ng/mm². Then, the wells were washed three times with 200 μL of DPBS to remove unbound proteins. Next, CHO-K1 N1-Gal4 cells in culture were detached with trypsin-EDTA 0.25% (Gibco) and manually counted. Appropriate dilutions were prepared in α-MEM media to ensure 30,000 CHO-K1 N1-Gal4 cells per well in 200 μL. Ligand-coated plates and reporter cell lines were incubated for 24 hours at 37° C. and 5% CO₂. On day two, CHO-K1 N1-Gal4 cells were washed with 200 μL DPBS, detached with 30 μL of trypsin-EDTA 0.25%, and quenched with 200 μL of α-MEM media. Finally, cells were resuspended and H2B-mCitrine signal was measured by flow cytometry (BD Accuri C6 plus). CHO-K1 N1-Gal4 and ligand-lacking wells were used as a negative control. The measurements represent the mean fluorescent intensity as percentage of Notch activation±s.d. of three biological replicates. Notch activation was reference-subtracted from a well coated with 0.5% BSA containing CHO-K1 N1-Gal4 cells. Gating strategies for this experiment are detailed in FIG. 13A.

Notch Activation with Ligand-Oriented Coupling on Streptavidin Plates

For CHO-K1 N1-Gal4 reporters, on day one, tissue culture 96-well plates (Coastar) were pre-treated with 100 μL of streptavidin at 10 μg/mL (VWR) for 1 hour at 37° C., washed three times with 200 μL DPBS (Corning), and blocked with 100 μL BSA 2% (VWR) for 1 hour at 37° C. Excess BSA was removed by washing three times with 200 μL DPBS (Corning), and 100 μL of DPBS containing biotinylated DLL4 proteins were coupled to streptavidin for 1 hour at 37° C. (2-fold serial dilutions starting at 50 nM). Non-coupled DLL4 proteins were removed by washing three times with 200 μL DPBS. Next, Notch reporter cells in culture were detached with trypsin-EDTA 0.25% (Gibco), and 30,000 cells in α-MEM media (200 μL) were added into individual wells. Assay plates were incubated for 24 hours at 37° C., and 5% CO₂. On day two, CHO-K1 N1-Gal4 cells were washed with 200 μL DPBS, detached with 30 μL of trypsin-EDTA 0.25%, and 200 μL of α-MEM media added. Cells were resuspended and Notch activation was measured as a function of fluorescence by flow cytometry (BD Accuri C6 plus). CHO-K1 N1-Gal4 cells and lacking-ligand wells were used as a negative control. The measurements represent the mean fluorescent intensity as percentage of Notch activation±s.d. of three biological replicates. Notch activation was reference-subtracted from a well coated with a non-related control protein (PA domain of RNF128).

For U2OS reporter cells, approximately 5×10E5 cells were plated into 6-well plates (Falcon) and let to adhere overnight at 37° C., 5% CO₂ in DMEM media (Cytiva). On day two, media was replaced with serum-free DMEM and cells transfected with luciferase vectors. Cationic lipoplexes were generated for 15 min at room temperature by mixing 10 μL of Lipofectamine 2000 (Invitrogen), 2.5 μg of Gal4-firefly luciferase reporter, and 50 ng of pRL-TK Renilla luciferase vector as internal control (ratio 1:5, Lipo:DNA). On day three, streptavidin plates were coupled with DLL4 ligands like described above using 10-fold serial dilutions starting at 50 nM, and transfected U2OS cells were detached with Trypsin-EDTA, washed with DPBS, and counted in the microscope. Twenty-thousand reporter cells were added to wells using 200 μL of DMEM media containing 2 μg/mL of doxycycline (Sigma) to induce Notch-Gal4 expression. Next, signaling plates were incubated for 24 hours at 37° C. and 5% CO₂. On day four, luciferase firefly and Renilla signals were determined with Dual-Glo luciferase assay system (Promega) using a GloMax luminometer (Promega). A luminescence ratio firefly:Renilla was determined for every well and normalized to the signal of a non-related control protein (PA domain of RNF128). Notch activation is represented as the mean fold-change over the control±s.d. of three independent biological replicates.

### Kinetics for Notch Activation Assay

96-well tissue culture plates (Coastar) were pre-coated using ligand-oriented coupling as described above with 50 nM of DLL4 biotinylated variants. CHO-K1 N1-Gal4 reporter cells were detached with trypsin-EDTA, washed with DPBS, and 200 μL of α-MEM media containing 30,000 cells were added into wells. Notch activation was analyzed as end-points for 0, 4, 8, 12, 16, 20, and 24-hour measurements. Desired time point wells containing CHO-K1 N1-Gal4 reporter cells were washed once with 200 μL DPBS, detached with trypsin-EDTA, and quenched with 200 μL of α-MEM media. Next, reporter cells were resuspended and analyzed by flow cytometry reading the fluorescence of H2B-mCitrine (BD Accuri C6 Plus). Every time point represents the mean fluorescence intensity of Notch activation as percentage+s.d. of three independent biological replicates. Notch activation was reference-subtracted from a well containing streptavidin, BSA and CHO-K1 N1-Gal4 cells.

Notch Activation in Co-Culture Format with HEK293T

On day one, U2OS reporter cells (signal receiver cells) were detached with trypsin-EDTA, washed with DPBS, and 5×10⁵ cells were added to 6-well plates (Falcon) for transfection with luciferase reporter vectors. On day two, U2OS cells were transfected as described above. On day three, signal receiver cells were detached with trypsin-EDTA, and 100 μL of DMEM media containing 20,000 cells were added to tissue culture 96-well plates (Coastar). In parallel, HEK293T-expressing WT DLL4 or Delta^{MAX} were detached with trypsin-EDTA, counted manually, and dilutions prepared such that 100 μL of DMEM containing 20,000 signal sender cells were added over signal receiver cells (cell ratio 1:1). Doxycycline (Sigma) at 2 μg/mL was used to induce Notch-Gal4 expression. On day four, luciferase firefly and Renilla signals were determined with Dual-Glo luciferase assay system (Promega) using a GloMax luminometer (Promega). A luminescence ratio firefly:Renilla was determined for every well and normalized to the signal of a well containing U2OS reporter cells and HEK293T non-expressing DLL4 ligands. Notch activation is represented as the mean fold-change over the control±s.d. of three independent biological replicates.

### Notch Signaling Assay Using Multiple Ligand-Presentation Formats

Notch activation was assayed in different formats (FIG. 14A) including (1) non-specific orientation (0.1 ng/mm² of Delta^{MAX} on well, FIG. 3A), (2) ligand-oriented coupling (10 nM of Delta^{MAX} on well, FIG. 3B), (3) magnetic beads coated with biotinylated Delta^{MAX} (ratio 1:20 reporter cells:beads, optimization of ratio is detailed in FIG. 14D), (4) HEK293T-expressing Delta^{MAX} (ratio 1:1, signal receivers:senders, optimization of ratio can be seen in FIG. 14C), and (5) yeast-expressing Delta^{MAX} (ratio 1:10, reporter:yeast, optimization of ratio is detailed in FIG. 14B). On day one, U2OS hNotch1-Gal4 cells were detached with trypsin-EDTA, counted in the microscope, and 5×10E5 cells/well transferred to 6-well tissue culture plates (Falcon). On day two, U2OS cells were transfected as described above. On day three, the different activation assays were set up at the same time. For ligand oriented-coupling, streptavidin plates were coated with 10 nM of Delta^{MAX} as described above. For non-specific adsorption, non-biotinylated Delta^{MAX} at 0.1 ng/mm was coated as described previously. For activation using streptavidin beads coated with biotinylated Delta^{MAX}, 200 mg of streptavidin beads (streptavidin MagneSphere paramagnetic particles, Promega) were washed 3 times with 200 μL of DPBS+0.1% BSA using a magnet (Miltenyi), and combined with 20 μg of biotinylated Delta^{MAX} for 1.5 hours at 4° C. Next, unbound Delta^{MAX} was washed out three times with 200 μL of DPBS+0.1% BSA, and 100 μL of complexed beads (pre-diluted 1/25 in DPBS) were added to wells. For co-culture activation with HEK293T-expressing Delta^{MAX}, cells were detached with trypsin-EDTA, quenched with DMEM media, washed with DPBS, and 100 μL containing 20,000 cells in DMEM were added to wells. For activation with yeast cells expressing Delta^{MAX} on surface, pYAL vector encoding for Delta^{MAX} (N-EGF5) was electroporated previously into yeast cells, and DLL4 expressed in SGCAA as previously described. Delta^{MAX} expression was induced 48 hours before Notch activation assay set up. One hundred microliters of yeast cells (2×10E6 cells/mL) in serum-free DMEM were added to the assay plate. Once all the wells contained the desired activation assays, U2OS reporter cells were detached with trypsin-EDTA, and 20,000 cells in 100 μL of DMEM were added per well. All the conditions were supplemented with 2 μg/mL of doxycycline to induce Notch1-Gal4 expression. The assay plate was incubated for 24 hours at 37° C. and 5% CO₂. On day four, media was aspirated off and Luciferase Dual-Glo kit (Promega) used to detect firefly and Renilla signals in a luminometer (GloMax, Promega). Negative controls were BSA 0.5% for non-specific adsorption, streptavidin and BSA 2% for ligand oriented-coupling, uncoated streptavidin beads for activation with magnetic beads, HEK293T non-expressing DLL4 for co-culture activation, and yeast expressing PDL-1 ECD for co-culture with yeast. The expression of Delta^{MAX} in HEK293T and yeast cells was confirmed by flow cytometry (BD Accuri C6 plus) following staining for 1 hour at 4° C. with anti-hDLL4 PE antibody using DPBS+0.5% BSA, and selection buffer, respectively (FIG. 14E). Notch activation is represented as the mean fold-change over the corresponding control±s.d. of three independent biological replicates.

Notch Activation with Streptavidin (SA)-Coated Polystyrene Beads

Biotinylated WT DLL4 or Delta^{MAX} (26 μg) were coated in 1.33 mg of streptavidin-coated polystyrene beads for 1 hour at 4° C. in DPBS+0.1% BSA. The beads were washed three times with 500 μL of DPBS+0.1% BSA before and after coating. DLL4 pre-coated beads were counted in the microscope and 100 μL containing 30,000 beads combined in ratio 1:1 with 100 μL of α-MEM with CHO-K1 N1-Gal4 reporter cell lines in 96-well tissue culture plates. The plate was incubated for 24 hours at 37° C., 5% CO₂, and Notch activation measure by flow cytometry. The data represent Notch activation as a function of mean fluorescent intensity±s.d. of three biological replicates.

Notch Inhibition Assays with Soluble Antagonists and WT DLL4 Coated Plates

For CHO-K1 N1-Gal4 reporter cells, on day one, biotinylated WT DLL4 was pre-coated in 96-well plates at 50 nM following the same protocol described above. Next, CHO-K1 N1-Gal4 cells were detached with trypsin-EDTA, washed, and diluted at 200,000 cells/mL in α-MEM media. Ten-fold serial dilutions of non-biotinylated DLL4 ligands (N-EGF5) were prepared in DPBS starting at 600 nM. Next, soluble inhibitors were mixed in ratio 1:1 with CHO-K1 N1-Gal4 200,000 cells/mL stocks for 15 min at room temperature to pre-block Notch receptors. Two hundred microliters of inhibition reactions containing 20,000 reporter cells were added to WT DLL4-coated wells, and plates incubated for 24 hours at 37° C. and 5% CO₂. When DAPT (gamma-secretase inhibitor, Sigma) and BB-94 (ADAM metalloprotease inhibitor, Sigma) were used, stock solutions were prepared at 100 μM in 8% DMSO. Appropriate DMSO controls were included to test Notch activation and cell viability. Final DMSO concentration in the experiment was kept below 1%. For comparisons of Delta^{MAX} with DAPT and BB-94, soluble inhibitors were serially diluted in DPBS using 9-folds, incubated with CHO-K1 N1-Gal4 reporter cells, and added to wells as described above. On day two, assay plates were washed with 200 μL of DPBS, CHO-K1 N1-Gal4 cells detached with trypsin-EDTA, quenched with 200 μL of α-MEM media, and Notch activation measured by flow cytometry (BD Accuri C6 Plus). The data represent the mean fluorescent intensity as percentage of Notch activation, relative to Notch activation in the absence of inhibitors±s.d. of three independent biological replicates.

For U2OS cells, on day one, 5×10⁵ cells were plated in 6-well tissue culture plates. On day two, U2OS cells were transfected as described above. On day three, 96-well plates (Coastar) were coated with streptavidin and biotinylated WT DLL4 at 50 nM as described above. Next, U2OS reporter cells were detached with trypsin-EDTA and diluted at 200,000 cells/mL in DMEM media. Ten-fold serial dilutions of soluble DLL4 variants starting at 6,000 nM were prepared in DPBS and used to pre-block reports cells following the same protocol described above. Two-hundred microliters of inhibition reactions were added to 96-well plates pre-coated with biotinylated WT DLL4 ligand at 50 nM, and assay plates incubated for 24 hours at 37° C. and 5% CO₂. On day fourth, luciferase signals were measured with Dual-Glo luciferase assay system (Promega) using a GloMax luminometer (Promega). Doxycycline at 2 μg/mL was maintained to ensure binding of soluble inhibitors to Notch-pan receptor. A ratio between firefly and Renilla luciferase was calculated, and Notch activation normalized to wells where inhibitors were non-added. The data represent fold-change of Notch activation as percentage, relative to Notch activation in the absence of inhibitors±s.d. of three independent biological replicates.

### Notch Inhibition Assay in Co-Culture Format

For inhibition of Notch ligand-expressing cell lines, on day one, U2OS Notch3 reporter cells (signal receivers) were detached with trypsin-EDTA, washed with DPBS, and added to 6-well plates. On day two, U2OS cells were transfected as described above. On day three, signal receiver cells were detached with trypsin-EDTA, and dilutions of 400,000 cells/mL were prepared in DMEM. On the one hand, soluble DLL4 variants were prepared at 6,000 nM in DPBS, combined in 1:1 dilution with signal receiver cells, and incubated for 15 min at RT. On the other hand, HEK293T-expressing WT DLL4, DLL1, JAG1, JAG2 were detached with trypsin-EDTA, washed with DPBS and 200,000 cells/mL stocks prepared in DMEM. One hundred microliters of both signal senders and pre-blocked signal receivers were combined in cell ratio 1:1 and incubated for 24 hours at 37° C., and 5% CO₂. On day four, luciferase firefly and Renilla signals were determined with Dual-Glo luciferase assay system (Promega) using a GloMax luminometer (Promega). Doxycycline at 2 μg/mL was kept for all the conditions. A luminescence ratio firefly:Renilla was determined for every well. Notch activation of each Notch3:ligand pair was normalized to the signal of Notch3 reporters in the presence of HEK293T lacking ligands. Each Notch signaling pair was set at 100% activation and soluble DLL4 treatments were relative to its corresponding pair. The data represent fold-change of Notch activation as percentage, relative to Notch activation in the absence of inhibitors±s.d. of three independent biological replicates.

For inhibition of CHO-K1 N1-Gal4 cells with DAPT and Delta^{MAX}, the experiment was set-up following the protocol described above with the exception that CHO-K1 N1-Gal4 reporters were incubated with HEK293T-expressing WT DLL4 for Notch activation. Three-fold serial dilutions of soluble Delta^{MAX} and DAPT were prepared in DPBS starting at 6,000 nM. For DAPT, a stock solution of 100 μM in 8% DMSO was prepared before serial dilutions in DPBS. Appropriate DMSO controls were also assayed for cytotoxicity. Final DMSO concentration in the wells was maintained below 1%. Thirty thousand signal senders and pre-blocked signal receivers (100 μL each) were incubated for 24 hours at 37° C. and 5% CO₂ before analysis by flow cytometry. Maximum Notch activation (100%) was set in the absence of soluble inhibitors and normalized to wells containing CHO-K1 N1-Gal4 cells and HEK293T lacking expression of WT DLL4. Mean fluorescence intensity is represented as percentage relative to Notch activation in the absence of inhibitors±s.d. of three independent biological replicates.

### Co-Culture Assays for T-Cell Activation and Proliferation

Functional assays for human CD8⁺ T cells were performed in co-culture with K562 cells expressing human CD32 (K32 cells) generated as described before7. Briefly, cells were γ-irradiated (100Gy) and washed twice with PBS before being loaded with OKT3 (0.0125-0.5 μg/mL, BioXcell) antibody at RT for 10 min. Human CD8⁺ T cells were negatively isolated from PBMC and labeled with 5 μM of Cell Trace Violet (Invitrogen) according to manufacturer's specifications. Next, CD8⁺ T cells were co-cultured with K32 cells at ratio 1:10 (T cell:K32). The frequency of proliferating T cells was determined after 96 hours by flow cytometry using a CytoFLEX II (Beckman Coulter). Zombie Green (Biolegend) was used for life/dead cell staining to discard dead cells from the analysis. A set of samples were additionally incubated with Golgi stop (BD) at 0.5 μL/well for 5 hours and washed with PBS for detection of intracellular IFNγ using anti-hIFNγ APC antibody (Biolegend). The frequency of IFNγ positive cells was analyzed by flow cytometry (CytoFLEX II, Beckman Coulter). Dead cells were excluded from the analysis by staining with Zombie green (Biolegend). The data represent the mean percentage of proliferating T cells or IFNγ⁺ T cells±s.e.m. of four independent biological replicates.

### Quantitative Real-Time PCR

Human CD8⁺ T cells were co-cultured with K32-DLL4 cells for 96 hours and positively enriched by MACS column (Invitrogen Kit). Total RNA was isolated from CD8⁺ T cells using TRIzol (Life Technologies). Reverse transcription reaction was performed using Verso cDNA Synthesis Kit (Thermo Scientific). Quantitative PCR reactions were prepared by using Bio-Rad SYBR green master mix and performed on an Applied Biosystems thermocycler (7900 HT). Relative expression was calculated using the ΔΔCt method and normalized to B2M levels. The data represent the mean quantity of mRNA (in-fold) for different T cell markers±s.e.m. of four independent biological replicates. Pair primers used were:

### Human β2-Microglobulin,

Forward:
(SEQ ID NO: 17)
5′-ATGAGTATGCCTGCCGTGTGA,
Reverse:
(SEQ ID NO: 18)
5′-GGCATCTTCAAACCTCCATG;
Human IFNγ,
Forward:
(SEQ ID NO: 19)
5′-GACCAGAGCATCCAAAAGAG,
Reverse:
(SEQ ID NO: 20)
5′-GGACATTCAAGTCAGTTACCGAATA;
Human Granzyme B,
Forward:
(SEQ ID NO: 21)
5′-CCCTGGGAAAACACTCACACA,
Reverse:
(SEQ ID NO: 22)
5′-GCACAACTCAATGGTACTGTCG;
Human Hes-4,
Forward:
(SEQ ID NO: 23)
5′-ACGGTCATCTCCAGGATGT,
Reverse:
(SEQ ID NO: 24)
5′-CGAGCGCGTATTAACGAGAG.

### Immunoblot Analysis

For detection of Notch activation in CD8⁺ T cells, following co-culture with K32 cell lines, CD8⁺ T cells were positively enriched by Magnisort Human CD8⁺ T cell positive selection Kit (Invitrogen). Nuclear fraction was isolated using NE-PER Nuclear and Cytoplasmic extraction kit (ThermoFisher). Equal protein amounts of nuclear cell lysates were run on 4-20% gradient Tris-glycine gels (Novex-Invitrogen), transferred to PVDF membranes using an iBlot Gel Transfer Device (ThermoFisher), and immunoblotted with antibodies anti-cleaved Notch1 (Val1744 rabbit mAb, Cell Signaling Technology, 1:1,000), anti-Notch2 (D76A6 rabbit mAb, Cell Signaling Technology, 1:1,000), and anti-β Actin (mouse mAb, Sigma, 1:20,000). Secondary antibodies anti-IgG (anti-rabbit or anti-mouse, GE Healthcare, 1:5,000) conjugated to horseradish peroxidase (HRP) were used for detection of proteins using ECL Western Blot Substrate Reagent (Thermo Fisher Scientific). Images were acquired using a Chemidoc Imaging System and analyzed with Image-Lab software (Bio-Rad).

For Notch activation in U2OS and MCF-7 cells, WT DLL4 and Delta^{MAX} biotinylated ligands were immobilized at 50 nM in 6-well streptavidin-coated plates following a scaled-up protocol of the one described before. After 24 hours of stimulation, cells were lysed in Laemmli serum buffer (40% glycerol, 4% SDS, 250 mM Tris-HCl, 0.02% bromophenol blue, and 20% β-mercaptoethanol). Cell lysates were heated at 100° C. for 5 min, centrifuged at 21,000×g for 10 min at 4° C., and supernatants stored at −20° C. Proteins were separated by SDS-PAGE (12% Mini-PROTEAN TGX Precast Protein Gels, Bio-Rad) and transferred to PVDF membranes using an iBlot2 Gel Transfer Device (Thermo Fisher Scientific). The membranes were blocked in 3% BSA+0.1% TBS-Tween. Primary antibodies were anti-cleaved Notch1 (Val1744 rabbit mAb, Cell Signaling Technology, 1:1,000), and β-Actin (rabbit pAb, Cell Signaling Technology, 1:1,000). Secondary antibody anti-Rabbit IgG conjugated to HRP (Goat mAb, Vector Laboratories, 1:8,000) was used for detection of proteins using SuperSignal West Pico PLUS Chemiluminescent Substrate (Thermo Fisher Scientific). Images were acquired using an Amersham Imager 600 (GE Healthcare), and contrast adjusted with a curves layer.

### ELISA Assay

Ten-fold serial dilutions of non-biotinylated WT DLL4 and Delta^{MAX} recombinant proteins (N-EGF5) were prepared in 100 μL of DPBS starting at a 13.31 ng/mm², and used to coat 96-well plates (Coastar) for 1 hour at 37° C. Next, the wells were washed three times with 200 μL of DPBS to remove unbound proteins and then blocked with 100 HL 2% BSA in DPBS (Thermo Scientific) for 1 hour at 37° C., followed by washing three times with DPBS. The wells were then incubated with an anti-6×His HRP-conjugated antibody (Bethyl, 1:5,000 dilution in DPBS+0.5% BSA) on a microplate tilter for 1 hour at RT. The wells were washed three times with DPBS, and 100 μL of Turbo TMB-ELISA (Thermo Scientific) substrate solution was added. After 15 min the reaction was stopped by addition of 100 μL 2M Hydrochloric acid and the absorbance was measured in a BioTek Synergy 2 microplate reader at 450 nm.

Human Pluripotent Stem Cell Culture and Differentiation into Neurons

H1 human pluripotent stem cells (hPSCs, WiCell, WA01) were used in this study and maintained. In brief, undifferentiated hPSCs were cultured in mTeSR Plus media (STEMCELL Technologies, 100-0276), with media changes every 1-2 days, on tissue culture plates that had been coated with Geltrex basement membrane matrix (Thermo Fisher). When undifferentiated hPSCs became partially to largely confluent, they were dissociated using Versene (Thermo Fisher), manually scraped off the plate, and then passaged as small clumps at a 1:6 ratio onto new Geltrex-coated plates in mTeSR Plus media (without ROCK inhibitor). To coat plates with Geltrex, a frozen Geltrex stock was thawed and diluted 1:100 in DMEM/F12 (Thermo Fisher), which was then added to the bottom of a tissue culture plate for at least 30 minutes at 37° C. The remaining Geltrex solution was then aspirated, leaving behind a thin film.

For neural differentiation, hPSCs were dissociated into single cells with Accutase (Thermo Fisher) and seeded at a density of ˜250,000 cells/cm² (i.e., 1×10⁶ cells/well of a 6-well plate) onto Geltrex-coated plates in mTeSR Plus media supplemented with thiazovivin (1 μM, a ROCK inhibitor to enhance cell survival; Tocris). The following day, mTeSR Plus media was aspirated, the cells were briefly washed with DMEM/F12, and neural differentiation media was added.

Neural differentiation of hPSCs was performed in two separate phases over 14 days. In the first phase, to differentiate hPSCs into neural progenitors for 7 days, a modification of a previously-reported method was used. However, in this study, differentiation conducted in Chemically Defined Media 2 (CDM2) media, whose composition has been described previously: 50% IMDM+GlutaMAX (Thermo Fisher)+50% F12+GlutaMAX (Thermo Fisher)+1 mg/mL polyvinyl alcohol (Sigma)+1% v/v chemically defined lipid concentrate (Thermo Fisher)+450 μM 1-thioglycerol (Sigma)+0.7 μg/mL recombinant human insulin (Sigma)+15 μg/mL human transferrin (Sigma)+1% v/v penicillin/streptomycin (Thermo Fisher). To create CDM2, polyvinyl alcohol was brought into suspension by gentle warming and magnetic stirring, and the media was sterilely filtered (through a 0.22 μm filter) prior to use. Throughout the first 7 days, differentiation media was changed every 24 hours. The second phase was to differentiate hPSC-derived neural progenitors into neurons for 7 days, from the day 8-14 interval. Neuronal differentiation media was modified from a previously-reported method⁹. From day 8-14, cells were differentiated in BDNF (20 ng/ml, PeproTech), GDNF (20 ng/ml, PeproTech), Forskolin (10 μM, Tocris), 2-Phospho-L-ascorbic acid (0.2 mM, Sigma-Aldrich), with the basal media compositions varying depending on the day of differentiation. On day 8, 25% Neurobasal media+75% CDM2 media was used; on day 9, 50% Neurobasal media+50% CDM2 media was used; on day 10, 75% Neurobasal media+25% CDM2 media was used; on days 11-14, 100% Neurobasal media was used. The composition of Neurobasal media was: Neurobasal basal media (Thermo Fisher)+1% v/v N2 (Thermo Fisher)+2% v/v B27 (Thermo Fisher)+1% v/v penicillin/streptomycin (Thermo Fisher), which was filtered through a sterilizing 0.22 μM filter before use. In summary, starting on day 8 of differentiation, Neurobasal media was added in an increasing ¼th increment on a daily basis. However, to emphasize, throughout the entire day 8-14 interval, cells were exposed to the same concentration of neuronal differentiation signals (BDNF, GDNF, Forskolin and 2-Phospho-L-ascorbic acid), although different basal medias were used on different days.

To test the effect of Notch signaling on neuronal differentiation, starting on day 8, hPSC-derived neural progenitors were treated with the aforementioned neuronal differentiation signals in the presence or absence of soluble Delta^{MAX} (0-1,000 nM), soluble wild-type DLL4 (0-1,000 nM), or DAPT (10,000 nM, Tocris).

### Quantitative PCR Analysis of Human Pluripotent Stem Cell-Derived Neurons

After differentiation of hPSCs into neurons as described above, gene expression was analyzed using RNA extraction, reverse transcription, and quantitative PCR (qPCR) as previously described. First, cells were lysed with RNeasy Lysis Buffer (Qiagen) and stored at −20° C. Cell lysates were then thawed, and RNA was extracted using the RNeasy Mini Plus Kit (Qiagen) as per the manufacturer's instructions and dissolved in H₂O. Next, 300 ng of RNA was reverse transcribed into complementary DNA (cDNA) using the High-Capacity cDNA Reverse Transcription Kit (Applied Biosystems) according to the manufacturer's protocol. Total cDNA was diluted 1:20 in H₂O and qPCR was performed with the SensiFAST SYBR Lo-ROX Kit (Bioline) using 10 μL qPCR reactions per well in a 384-well plate. Each individual reaction contained 5.0 μL 2× SensiFAST SYBR qPCR Master Mix+4.6 μL cDNA+0.4 μL of 10 μM primer stock (5 μM forward+5 μM reverse primers).

After qPCR plates were prepared by arraying sample-specific cDNAs and gene-specific primers, they were sealed and briefly centrifuged (for 1 minute). 384-well qPCR plates and their adhesive sealing sheets were obtained from Thermo Fisher. qPCR plates were ran on a QuantStudio 5 qPCR machine (Thermo Fisher) with the following cycling parameters: initial dissociation (95° C., 2 mins) followed by 40 cycles of amplification and SYBR signal detection (95° C. dissociation, 5 seconds; 60° C. annealing, 10 seconds; followed by 72° C. extension, 30 seconds), with a final series of steps to generate a dissociation curve at the end of each run. During qPCR data analysis, the fluorescence threshold to determine Ct values was set at the linear phase of amplification. Expression of all genes was normalized to the levels of the reference gene YWHAZ. The data represent the mean quantity of mRNA for different markers±s.e.m. of four independent biological replicates. Pair primers used were:

DCX,
Forward:
(SEQ ID NO: 25)
5′-AGGGCTTTCTTGGGTCAGAGG,
Reverse:
(SEQ ID NO: 26)
5′-GCTGCGAATCTTCAGCACTCA;
MAPT,
Forward:
(SEQ ID NO: 27)
5′-CCAAGTGTGGCTCATTAGGCA,
Reverse:
(SEQ ID NO: 28)
5′-CCAATCTTCGACTGGACTCTGT;
SNAP25,
Forward:
(SEQ ID NO: 29)
5′-ACCAGTTGGCTGATGAGTCG,
Reverse:
(SEQ ID NO: 30)
5′-CAAAGTCCTGATACCAGCATCTT;
SOX2,
Forward:
(SEQ ID NO: 31)
5′-TGGACAGTTACGCGCACAT,
Reverse:
(SEQ ID NO: 32)
5′-CGAGTAGGACATGCTGTAGGT;
SYT1,
Forward:
(SEQ ID NO: 33)
5′-GCTGCTGGTAGGGATCATTCA,
Reverse:
(SEQ ID NO: 34)
5′-GTTTTTCGGTGGACTTTTGTCTC;
TBR1,
Forward:
(SEQ ID NO: 35)
5′-ACAATTTTCCTGACTCCAAGGAC,
Reverse:
(SEQ ID NO: 36)
5′-ACTGTGACGAAGCTCAGAGAC;
VGLUT2,
Forward:
(SEQ ID NO: 37)
5′-GGGAGACAATCGAGCTGACG,
Reverse:
(SEQ ID NO: 38)
5′-TGCAGCGGATACCGAAGGA;
YWHAZ,
Forward:
(SEQ ID NO: 39)
5′-GAGCTGGTTCAGAAGGCCAAAC,
Reverse:
(SEQ ID NO: 40)
5′-CCTTGCTCAGTTACAGACTTCATGCA.

### Reporting Summary

Further information on research design is available in the Nature Research Reporting Summary linked to this article.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A method of generating a cell of the T cell lineage comprising (a) culturing a sample comprising stem cells or progenitor cells with an engineered Notch ligand conjugated to a suspension support and (b) isolating cells of the T cell lineage,
wherein the engineered Notch ligand comprises a conservative amino acid substitution at a residue corresponding to residues 28, 107, 143, 194, and 206 as set forth in SEQ ID NO: 1 or SEQ ID NO: 7 and further comprising at least one conservative amino acid substitution at residues 256, 257, 271, 280, 301, and 305 as set forth in SEQ ID NO: 1 or SEQ ID NO: 7.

2. The method of claim 1, wherein the suspension support is a particle or a microbead.

3. The method of claim 1, wherein the stem cells or progenitor cells with the engineered Notch ligand are cultured in suspension.

4. The method of claim 1, wherein the stem cells are selected from hematopoietic stem/progenitor cells (HSPCs), embryonic stem cells and induced pluripotent stem cells (iPSCs).

5. The method of 4, wherein the stem cells are CD34⁺ or CD34⁺ CD38^{-/lo} HSPCs.

6. The method of claim 1, wherein the stem cells are CD34⁺ hematopoietic precursor cells.

7. The method of claim 1, wherein the cells of the T cell lineage are progenitor T (proT) cells.

8. The method of claim 7, wherein the stem cells or progenitor cells are human cells and the proT cells have the phenotype CD34⁺ CD7⁺ or CD7⁺ CD5⁺ CD1a⁻.

9. (canceled)

10. The method of claim 1, wherein the cells of the T-cell lineage are CD4⁺CD8⁺ double positive cells, CD4+CD8+CD3+ double positive cells, CD8⁺CD3⁺ single positive cells or CD4⁺CD3⁺ single positive cells.

11. The method of claim 1, wherein the stem cells or progenitor cells are cultured in stromal cell-free media.

12. The method of claim 1, wherein the stem cells or progenitor cells are cultured with at least one T cell co-stimulatory molecules attached to a suspension support, wherein the at least one T cell co-stimulatory molecule is VCAM1.

13. The method of claim 1,
wherein the substitution at residue 28 comprises a glysine to serine substitution (G28S),
wherein the substitution at residue 107 comprises a phenylalanine to leucine substitution (F107L),
wherein the substitution at residue 143 comprises a isoleucine to phenylalanine substitution (I143F),
wherein the substitution at residue 194 comprises a histidine to tyrosine substitution (H194Y),
wherein the substitution at residue 206 comprises a leucine to proline substitution (L206P),
wherein the amino acid at residue 256 comprises a histidine, tyrosine, phenylalanine, leucine, asparagine, isoleucine, valine, or aspartic acid (H256Y, H256F, H256L, H256N, H256I, H256V, or H256D),
wherein the amino acid at residue 257 comprises a proline, histidine, leucine, isoleucine, threonine, asparagine, tyrosine, serine, or phenylalanine (N257P, N257H, N257L, N257I, N257T, N257Y, N257S, or N257F),
wherein the amino acid at residue 271 comprises a leucine, proline, histidine, asparagine, threonine, or isoleucine (T271L, T271P, T271H, T271N, or T271I),
wherein the amino acid at residue 280 comprises a phenylalanine, leucine, tyrosine, or histidine (F280Y, F280L, or F280H),
wherein the amino acid at residue 301 comprises a serine, asparagine, arginine, or histidine (S301H, S301N, or S301R), and
wherein the amino acid at residue 305 comprises a glutamine, proline, arginine, or leucine (Q305P, Q305R, or Q305L).

14-18. (canceled)

19. The method of claim 1, wherein the substitution at residue 256 comprises a histidine to tyrosine substitution (H256Y).

20. (canceled)

21. The method of claim 1, wherein the substitution at residue 257 comprises an asparagine to proline substitution (N257P).

22. (canceled)

23. The method of claim 1, wherein the substitution at residue 271 comprises a threonine to leucine substitution (T271L).

24. (canceled)

25. The method of claim 1, wherein the substitution at residue 280 comprises a phenylalanine to tyrosine substitution (F280Y).

26. (canceled)

27. The method of claim 1, wherein the substitution at residue 301 comprises a serine to histidine substitution (S301H).

28. The method of claim 1, wherein the substitution at residue 301 comprises a serine to arginine substitution (S301R).

29. (canceled)

30. The method of claim 13, wherein the substitution at residue 305 comprises a glutamine to proline substitution (Q305P).

31. The method of claim 1, wherein the engineered Notch ligand comprises the amino acid sequence SEQ ID NO: 3, SEQ ID NO: 4, SEQ ID NO: 5, SEQ ID NO: 6, SEQ ID NO: 9, SEQ ID NO: 10, SEQ ID NO: 11, or SEQ ID NO: 12.

32-36. (canceled)
